(19) United States
(12) Patent Application Publication
(10) Pub. No.: US 2023/0393120 A1
(43) Pub. Date: Dec. 7, 2023

(54) A HUMAN VASCULAR INJURY-ON-A-CHIP MODEL OF HEMOSTASIS

(71) Applicant: THE TRUSTEES OF THE UNIVERSITY OF PENNSYLVANIA, Philadelphia, PA (US)

(72) Inventors: Keon Woo KWON, Penn Valley, PA (US); Dongeun HUH, Villanova, PA (US); Lawrence F. BRASS, Bala Cynwyd, PA (US); Izmarie POVENTUD-FUENTES, Houston, TX (US)

(21) Appl. No.: 18/250,764

(22) PCT Filed: Oct. 29, 2021
(86) PCT No.: PCT/US2021/072132

§ 371 Date: Apr. 27, 2023

Related U.S. Application Data
(60) Provisional application No. 63/107,978, filed on Oct. 30, 2020.

(51) Int. Cl. G01N 33/50; C12N 5/071; C12M 3/06; C12M 3/00; C12M 1/34

(52) U.S. Cl. CPC G01N 33/5082; C12N 5/0691; C12M 23/16; C12M 21/08; C12M 41/46; C12N 2513/00; C12N 2537/10

(57) ABSTRACT

The present subject matter relates to techniques for mimicking the hemostasis microenvironment and predicting the effects of drugs on hemostasis. The disclosed system can include a top layer including a plurality of top rails, and a bottom layer including a plurality of bottom rails, wherein the top layer and the bottom layer are configured to be coupled, wherein the plurality of top rails and bottom rails are configured to form a plurality of channels comprising an intravascular channel configured to circulate a first solution, an extravascular channel configured to circulate a second solution, and a vessel wall channel including a tissue factor in a hydrogel.

### RELATED APPLICATIONS

This application claims priority to and the benefit of U.S. patent application 63/107,978, “Human Vascular Injury-On-A-Chip Model Of Hemostasis” (filed Oct. 30, 2020), the entirety of which application is incorporated herein by reference for any and all purposes.

### GOVERNMENT SUPPORT

This invention was made with government support under HL127720, HL120846 and HL040387 awarded by the National Institutes of Health and 1548571 awarded by the National Science Foundation. The government has certain rights in the invention.

### BACKGROUND

Hemostasis can be initiated by the adhesion of platelets to collagen fibrils exposed within the vessel wall. As bleeding continues, escaping blood can contact tissue factor in the matrix surrounding the blood vessel, triggering thrombin generation, further platelet activation, and fibrin accumulation. The growth of this temporary plug can be regulated by serpins and protein C, which can limit thrombin formation and activity, and by molecules released by endothelial cells. Concurrently, the growing mass of platelets and fibrin undergoes contractive deformation, helping to contain agonists generated at the site of injury and hindering the exchange of soluble factors. These events can decrease coagulation reactions and limit the extent of platelet activation, eventually giving rise to an optimal hemostatic plug that seals the injury in the vessel wall.

Certain techniques and systems were developed to investigate hemostasis by mimicking hemostasis environments. For example, certain animal models were developed using mouse models (e.g., tail clip assay). Such models are limited by differences in the size and number of platelets, their sensitivity to biochemical mediators of hemostasis, and the distribution of coagulation factors. Certain In vitro models were developed using human cells as an alternative to in vivo models. However, these in vitro models can suffer from limited capacity to reproduce the dynamic microenvironment in which the hemostatic response normally occurs.

Therefore, there is a need for improved techniques that can be used for mimicking the hemostasis microenvironment and predicting the impact of new drugs on hemostasis.

### SUMMARY

The disclosed subject matter provides techniques for mimicking the hemostasis microenvironment and predicting the effects of drugs on hemostasis. The disclosed subject matter provides systems that can be physiologically relevant to the human vascular system and methods for drug screening.

In an example embodiment, the disclosed system can include a top layer including a plurality of top rails and a bottom layer including a plurality of bottom rails. The top layer and the bottom layer can be configured to be coupled. The top rails and bottom rails can be configured to form a plurality of channels. The channels can include an intravascular channel configured to circulate a first solution, an extravascular channel configured to circulate a second solution, and a vessel wall channel including a tissue factor in a hydrogel.

In certain embodiments, the hydrogel can include an endothelial cell for generating an endothelial monolayer. In non-limiting embodiments, the endothelial cell is HUVEC.

In certain embodiments, the disclosed system can receive a needle that can be inserted between the intravascular channel and the extravascular channel for generating puncture injury on the endothelial monolayer. In non-limiting embodiments, the hydrogel can be a collagen hydrogel.

In certain embodiments, the disclosed system can further include an inlet port and an outlet port for accessing the plurality of channels. In non-limiting embodiments, the plurality of channels can include a microchannel. In some embodiments, the plurality of top rails and bottom rails can be microfabricated rails.

In certain embodiments, the first solution can include recalcified blood. In non-limiting embodiments, the second solution can include an HBSS buffer.

The disclosed subject matter also provides methods for drug screening. The method can include seeding an endothelial cell in a hydrogel using a device comprising a plurality of channels comprising an intravascular channel, an extravascular channel, and a vessel wall channel, culturing the endothelial cell by adding culture medium to the intravascular channel and the extravascular channel, forming an endothelial monolayer in the vessel wall channel, adding a target drug into the culture medium, and measuring platelet deposition. The hydrogel can be located in the vessel wall channel and include tissue factor.

In certain embodiments, the method can further include measuring fibrin deposition. In non-limiting embodiments, the method can include measuring platelet and fibrin deposition before adding the target drug. In some embodiments, the method can include comparing the platelet and fibrin deposition before adding the target drug with the measured platelet and fibrin deposition after adding the target drug. In non-limiting embodiments, the target drug can be added through an inlet port of the device.

In certain embodiments, the method can include generating puncture injury on the endothelial monolayer.

In certain embodiments, the target drug can be an anticoagulant drug, an antiplatelet drug, or a combination thereof. In non-limiting embodiments, the endothelial cell can be HUVEC. In some embodiments, the hydrogel is a collagen hydrogel.

The disclosed subject matter will be further described below.

### BRIEF DESCRIPTION OF THE DRAWINGS

FIGS. 1A-1J provide photographs and diagrams of an example system for modeling of hemostasis after a penetrating injury in accordance with the disclosed subject matter.

FIGS. 2A-2I provide graphs and images showing the formation of platelet and fibrin-rich hemostatic plugs using an example system after a puncture injury in accordance with the disclosed subject matter.

FIGS. 3A-3G provide confocal images and graphs showing images showing the characterization of platelet activation and fibrin formation in accordance with the disclosed subject matter.

FIGS. 4A-4D provide diagrams and images showing graphs showing In vitro and In vivo comparison of hemostatic plugs in accordance with the disclosed subject matter.

FIGS. 5A-5F provide graphs and images showing drug testing in the vascular injury-on-a-chip in accordance with the disclosed subject matter.

FIGS. 6A-6D provide diagrams showing designs of an example system in accordance with the disclosed subject matter.

FIG. 7 provides a diagram showing an example device for flow control in the vascular injury-on-a-chip in accordance with the disclosed subject matter.

It is to be understood that both the foregoing general description and the following detailed description are exemplary and are intended to provide further explanation of the disclosed subject matter.

### DETAILED DESCRIPTION

The disclosed subject matter provides techniques for mimicking the hemostasis microenvironment and predicting the effects of drugs on hemostasis. The disclosed subject matter provides systems that can be physiologically relevant to the human vascular system and methods for drug screening. The disclosed subject matter can be used for emulating and probing the inner workings of the hemostatic response. The disclosed subject matter can also be used for assessing therapies to restore the hemostatic balance. The disclosed subject matter can also be used for modeling thrombosis and other hematological disorders that involve abnormal changes in blood. The disclosed subject matter can also be used for screening the potential of pharmaceuticals, indwelling biomedical devices, and chemicals to induce bleeding and thrombosis and/or to cause changes in hemostasis.

Unless otherwise defined, all technical and scientific terms used herein have the same meaning as commonly understood by one of ordinary skill in the art. In case of conflict, the present document, including definitions, will control. Certain methods and materials are described below, although methods and materials similar or equivalent to those described herein can be used in the practice or testing of the presently disclosed subject matter. All publications, patent applications, patents, and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude additional acts or structures. The singular forms “a,” “an,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, and up to 1% of a given value. Alternatively, particularly with respect to biological systems or processes, the term can mean within an order of magnitude, within 5-fold, and within 2-fold, of a value.

In certain embodiments, the disclosed subject matter provides a system for mimicking the hemostasis microenvironment and predicting the effects of drugs on hemostasis. An example system can include a top layer and a bottom layer. The top layer and the bottom layer can be configured to be coupled. The term “coupled,” as used herein, refers to the connection of a device component to another device component by techniques known in the art. The type of coupling used to connect two or more device components can depend on the scale and operability of the device.

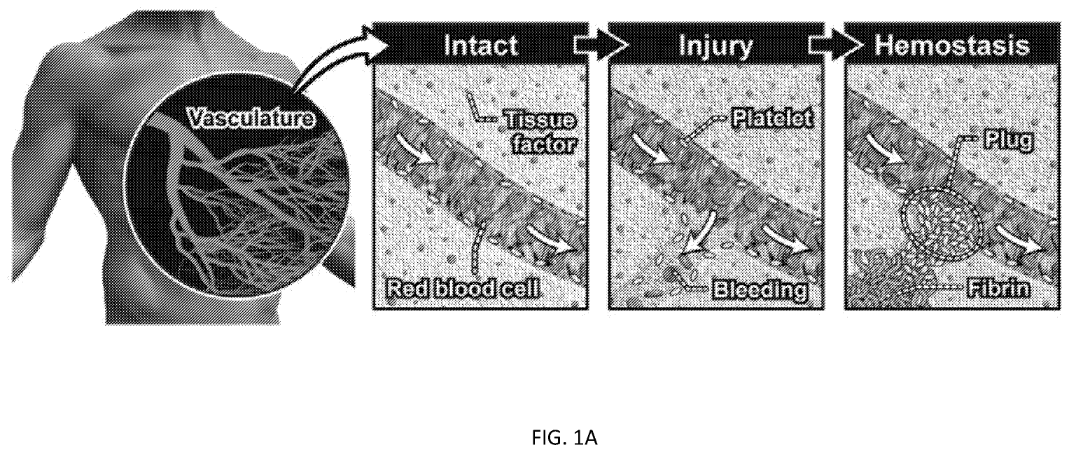

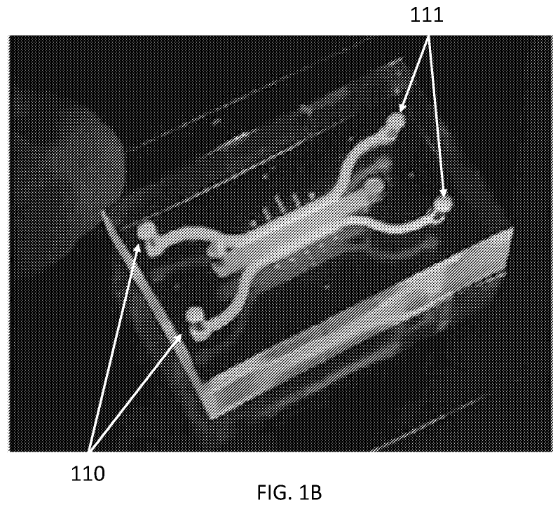

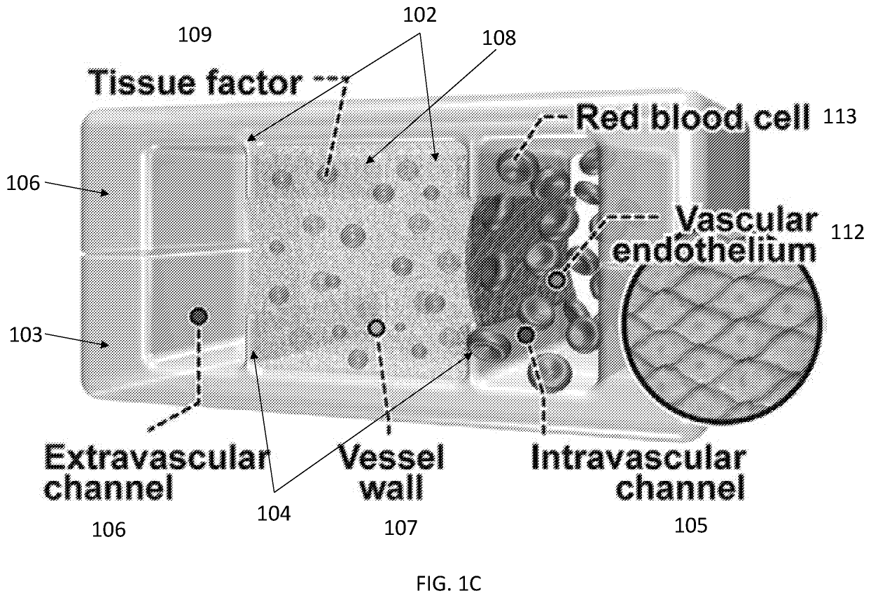

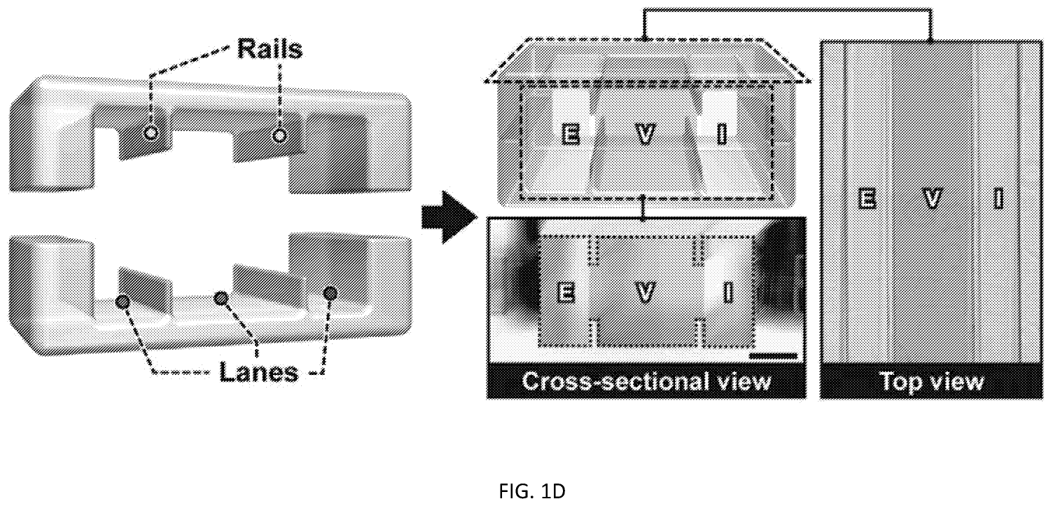

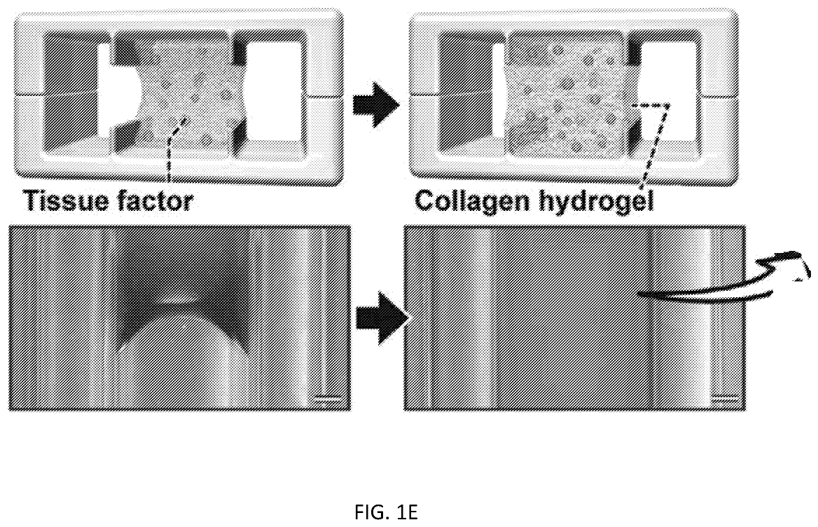

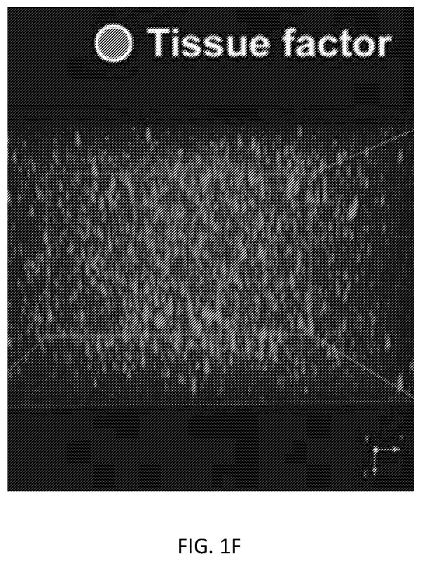

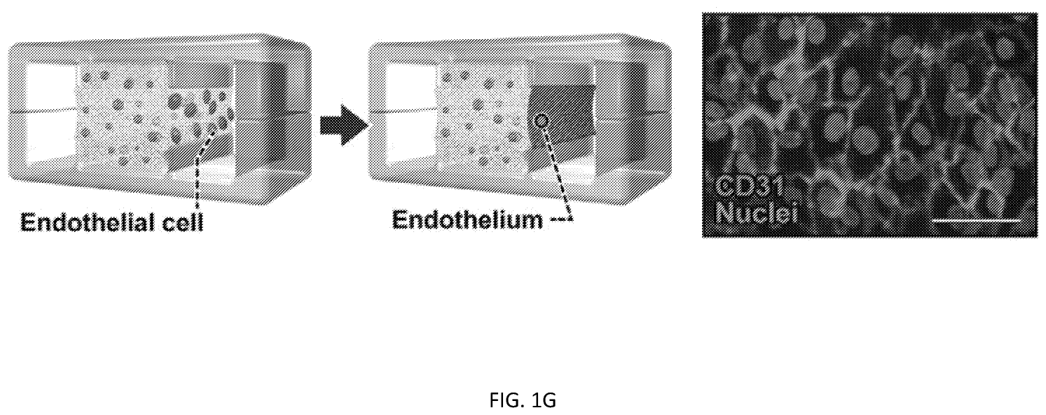

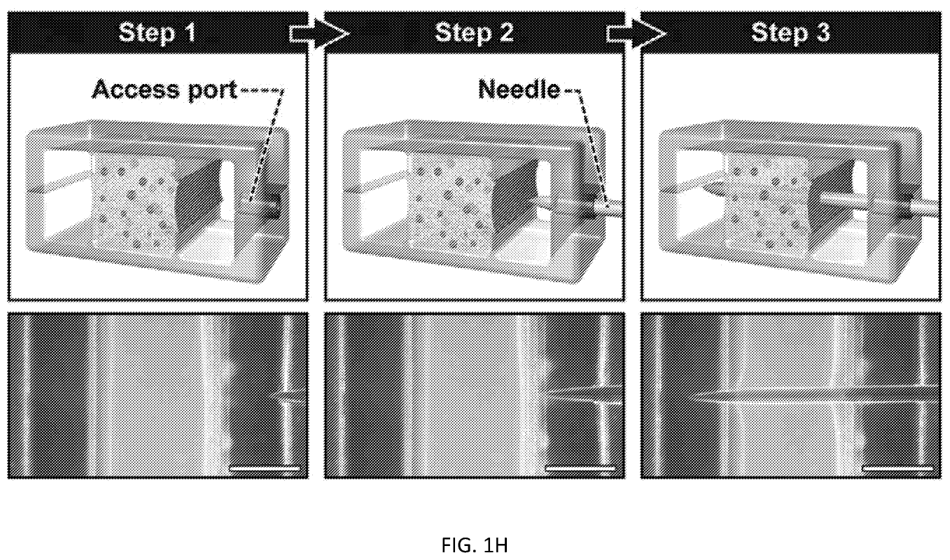

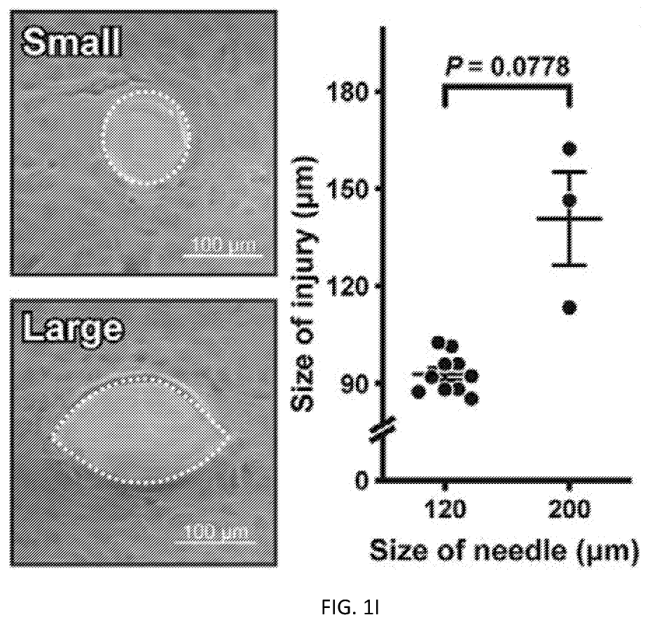

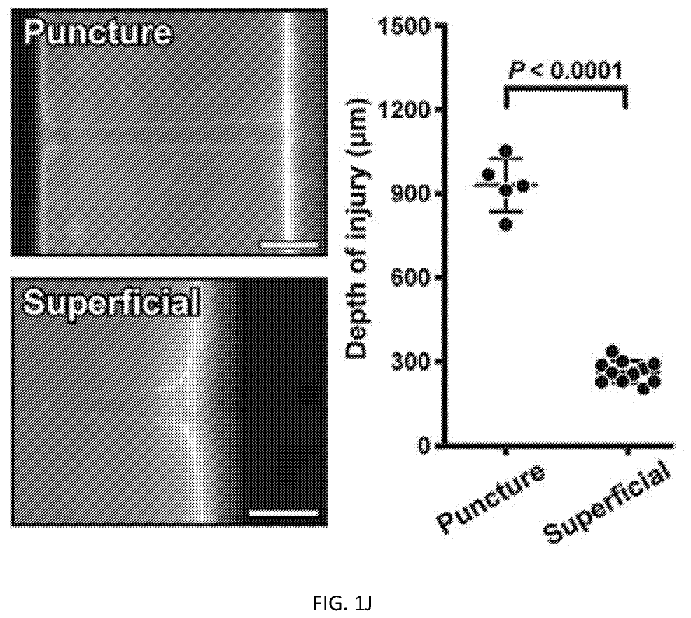

In certain embodiments, as shown in FIG. 1C, the top layer 101 can include top rails 102, and the bottom layer 103 can include bottom rails 104. In non-limiting embodiments, the rails can be patterned on the surface of the layers by photolithographic and soft lithographic techniques. For example, Poly(dimethylsiloxane) (PDMS) base can be mixed with a curing agent (e.g., at a weight ratio of 10:1), degassed to remove air bubbles, and poured onto the masters. The Masters can be SU-8 masters containing the rails and the microfluidic channel features. After baking (e.g., at 65° C. for 3 hours), the cured polymer can be peeled from the masters to generate two PDMS layers embossed with microchannel features. In some embodiments, the channels can be fabricated in other elastomers, thermoplastics, metals, papers, glasses, silicon, or woods. In some embodiments, the top layer and the bottom layer can be identical layers that include three lanes divided by two rails.

In certain embodiments, the size of rails can range from about 0.01 mm to about 1 mm.

In certain embodiments, the top rails and bottom rails can form a plurality of channels. For example, as shown in FIG. 1C, each channel can be separated by the rails running along the length of the channel. Alignment and bonding of the two layers can produce a sealed microdevice containing microchannels (e.g., with cross-sectional dimensions of 1 mm (width)×1 mm (height) flanked by two microchannels that measured 500 pm (width)×1 mm (height)). In non-limiting embodiments, the channels can be interconnected parallel microchambers, which can emulate the physiological compartmentalization of vascular tissue. In some embodiments, the channels can be a microchannel and/or the trails can be microfabricated rails.

In certain embodiments, as shown in FIG. 1C, the channels defined by the rails can include an intravascular channel 105, an extravascular channel 106, and a vessel wall channel 107. In non-limiting embodiments, the vessel wall channel (e.g., the middle chamber) can be configured to house a 3D hydrogel 108 (e.g., collagen hydrogel) containing homogeneously distributed tissue factor 109 to model the deformable, procoagulant wall of a blood vessel. The middle chamber can open to the side channels through the gap between the upper and lower rails. In non-limiting embodiments, the disclosed system can include an inlet port 110 and an outlet port 111 for accessing the channels and controlling fluids in the channels. For example, the three compartments can be equipped with their dedicated access ports for independent fluidic control.

In certain embodiments, the hydrogel can include tissue factor. For example, a collagen hydrogel precursor solution can be mixed with lipidated tissue factor and injected into the vessel channel. In certain embodiments, the hydrogel can include spatially graded tissue factors. In certain embodiments, the hydrogel can contain fiborblasts, pericytes, muscle cells, and/or other cell types found in the sub-endothelial compartment of blood vessels. In certain embodiments, the hydrogel can contain engineered ECM materials, perfusable blood vessels, and/or engineered micro and nanomaterials. The injected solution can be pinned at the rails by surface tension, which can prevent the spillage of the solution into the side channels. In non-limiting embodiments, the hydrogel can include an endothelial cell for generating an endothelial monolayer 112. For example, human umbilical vein endothelial cells (HUVECs) can be cultured on the exposed collagen surface for forming an endothelial barrier with structural integrity. In certain embodiments, the hydrogel can include epithelial cells and other types of parenchymal cells found in and around blood vessles, ducts, the respiratory tract, the gastrointestinal tract, and the reproductive tract.

In certain embodiments, the hydrogel can include an active agent. For example, the hydrogel can include a tumor necrosis factor (TNF)-a. In certain embodiments, the hydrogel can include ligh- and chemical-actuated materials. In certain embodiments, the hydrogel can contain mechanically and/or electrically addressable materials. In certain embodiments, the hydrogel can contain orgnaoids, tissue explants, spheroids, and other three-dimensional multicellular structures.

In certain embodiments, the intravascular channel can be configured to circulate a first solution and/or perfuse the hydrogel with the first solution. For example, the intravascular channel can be used to represent the intravascular compartment in which the human vascular endothelium can be generated on the exposed hydrogel surface and perfused with whole blood 113 at a venous shear rate to recapitulate the hemodynamic environment of the native vascular system. In certain embodiments, a solution can include chemicals, micro/nano particles, cells, gas bubbles, in-dwelling biomedical devices, and/or miniaturized sensors. In non-limiting embodiments, the solution can be added to the intravascular channel. In non-limiting embodiments, an active agent and/or a drug can be added into the intravascular channel through the inlet port.

In certain embodiments, the extravascular channel can be configured to circulate a second solution and/or collect certain compositions. For example, the left microchannel can be served as an extravascular compartment into which blood can escape when a hole is punched through the vessel wall. In non-limiting embodiments, the second solution can be an HBSS buffer. In non-limiting embodiments, the second solution can be blood, plasma, or interstial fluid. In non-limiting embodiments, an active agent and/or a drug can be added into the extravascular channel through the inlet port.

In certain embodiments, the disclosed system can simulate bleeding caused by penetrating vascular injury. For example, the disclosed system can include a cylindrical access port at the side of the disclosed system to permit direct entry into the channels. To create the injury, a flexible needle can be inserted through the access port. The needle can be guided into the intravascular channel, piercing the endothelial barrier and the collagen hydrogel construct, and reaching the extravascular channel on the other side. Withdrawing the needle generated an open puncture wound across the vessel wall. In non-limiting embodiments, the size of the endothelial injury can be adjusted by changing the diameter of the needle. The diameter can range from 100 nm to 1 cm. In some embodiments, the injury depth can be adjusted. For example, full injuries can be created by penetrating the entire vascular construct, while superficial injuries can be created by penetrating partial-thickness (e.g., 25% of the full thickness) of the vessel wall. In certain embodiments, the injury can be generated by using other mechanical tools such as scalpels, biopsy punches, small ballistics, stretching, torsion, compression, or their combinations. In certain embodiments, the injury can be caused by using chemicals that damage the vascular tissue. In certain embodiments, the injury can be caused by using an electrical device such as high voltage, high current, and high electrical field. In certain embodiments, the injury can be caused by magnectic forces. In certain embodiments, the injury can be caused by an optical device (e.g., laser).

In certain embodiments, the disclosed system can simulate the formation of hemostatic plugs. To generate hemostatic plugs, the intravascular channel can be perfused with human whole blood recalcified in the presence of corn trypsin inhibitor, and a buffer solution (e.g., HBSS) can be pulled through the extravascular channel. When the vessel wall is punctured with the needle, the difference in the rates of flow in the intravascular and extravascular channels can produce a pressure drop across the vascular construct (i.e., in the vessel channel), promoting the blood in the intravascular compartment to escape into the extravascular channel. With the disclosed penetrating injury, platelets can adhere to the surface of the exposed hydrogel (e.g., collagen matrix) and begin to form aggregates over time. In non-limiting embodiments, these deposits were initially observed at the ends of the puncture, but with time, platelet accumulation became pronounced and extended throughout the injury as the aggregates grew larger. Fibrin deposition as filamentous structures can be induced within the injury channel, and the simultaneous accumulation of platelet and fibrin can produce hemostatic plugs that can stop blood loss (e.g., within 10 minutes of injury).

In certain embodiments, the disclosed system can be used to study thromobosis. In certain embodiments, the disclosed system can be used to study bleeding disorders.

In certain embodiments, the disclosed system can be an optically transparent system. For example, the optical transparency of the disclosed system can allow a user to observe the dynamic process of bleeding and measure the kinetics of platelet accumulation in real-time. In non-limiting embodiments, the intravascular and extravascular compartments can be excised to expose the vessel wall (e.g., in the vessel well chamber). By exposing the vessel wall to the external environment, direct visualization (e.g., SEM imaging) and modification (e.g., harvesting the hydrogel) of the intravascular and extravascular surfaces around the injury site can be achieved.

In certain embodiments, the disclosed subject matter provides a method for drug screening. The method can include seeding an endothelial cell in a hydrogel using the disclosed system, culturing the endothelial cell by adding culture medium to the intravascular channel and the extravascular channel, forming an endothelial monolayer in the vessel wall channel, adding a target drug into the culture medium, and measuring platelet deposition. In non-limiting embodiments, the target drug can be an anticoagulant drug, an antiplatelet drug, or a combination thereof. As anticoagulants and antiplatelet agents can increase the risk of bleeding, the disclosed system (e.g., vascular injury-on-a-chip model) can be used to evaluate these adverse effects and offer a complementary approach to animal studies. For example, the disclosed system can be perfused with whole blood containing Hirudin and eptifibatide. A clinically relevant concentration of Hirudin (e.g., about 4 μg/ml) and eptifibatide (about 100 μM) can be administered.

In certain embodiments, the method can further include fibrin deposition. The measured platelet and fibrin deposition can indicate the level of injury. For example, by comparing the platelet and fibrin deposition before an injury with the measured platelet and fibrin deposition after the injury, the effects of the injury can be evaluated. In non-limiting embodiments, the injury can be a penetrating vascular injury and/or a drug-induced injury. For example, the effects of the target drug can be measured by comparing the platelet and fibrin deposition before adding the target drug into the disclosed system with the measured platelet and fibrin deposition after adding the target drug. In certain embodiments, the disclosed system can be used to model and assess the deformation of blood vessels due to the formation of hemstatic plugs. In certain embodiments, the disclosed system can be used to assess the deformation of the blood vessels due to injury, bleeding, and biomedical implants.

The disclosed subject matter provides microengineering techniques for modeling hemostasis. The disclosed system can replicate the living endothelium, multilayered microarchitecture, and procoagulant activity of human blood vessels. The disclosed system can be used as an injury model in which a microneedle can be actuated with spatial precision to simulate penetrating vascular injuries. The disclosed system can simulate thrombin-driven accumulation of platelets and fibrin, the formation of platelet- and fibrin-rich hemostatic plug that halts blood loss, and matrix deformation driven by platelet contraction for wound closure. In non-limiting embodiments, the disclosed subject matter can be used for drug screening as a preclinical model of hematological disorders.

### EXAMPLE 1

### A Human Vascular Injury-on-a-Chip Model of Hemostasis

Hemostasis can be a tightly-regulated process. When a closed, high-pressure vascular system is penetrated, bleeding continues until a stable hemostatic thrombus seals the breach and prevents further blood loss (FIG. 1A). The disclosed subject matter provides techniques for microengineering of biologically active three-dimensional (3D) extravascular tissue and modeling vascular injury (e.g., Hemostasis).

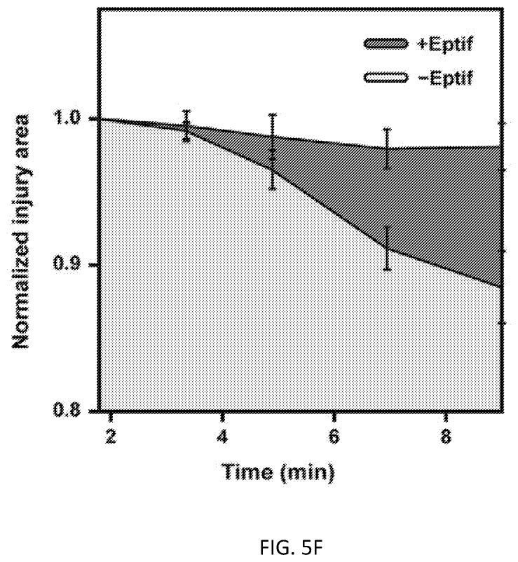

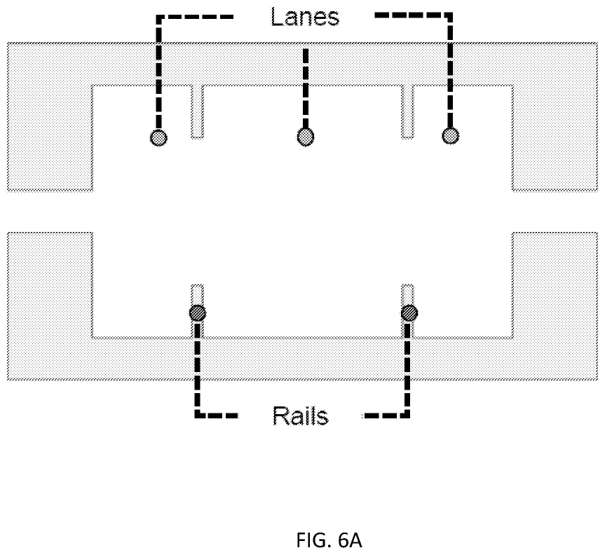

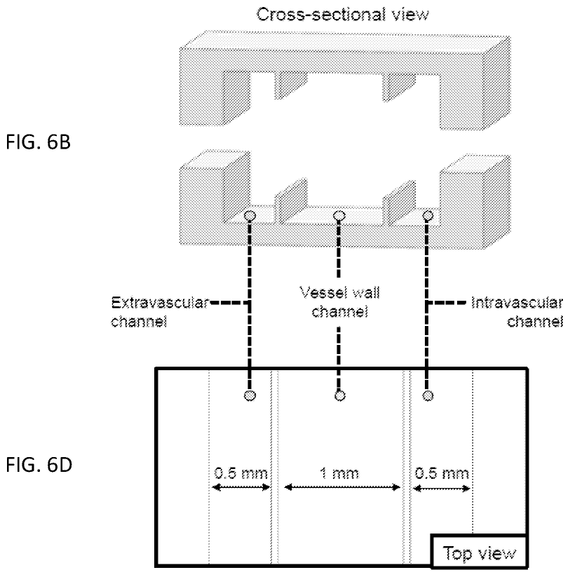

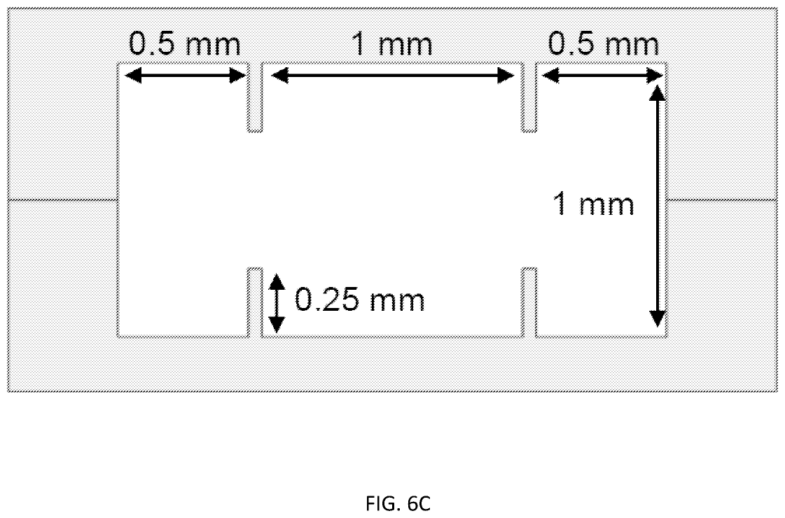

Microengineering of human blood vessel-on-a-chip: a microphysiological model of a human blood vessel was created in an optically transparent microfluidic device (FIG. 1B, FIGS. 6A-6D).

FIGS. 1A-1J shows a human vascular injury-on-a-chip for in vitro modeling of hemostasis after a penetrating injury. When an injury occurs in a blood vessel, a hemostatic plug stops bleeding (FIG. 1A). FIG. 1B shows an image of a blood vessel-on-a-chip microdevice. FIG. 1C shows the system designed to model three distinct tissue compartments at the injury site. Microfabricated rails on the top and bottom channel walls divide the assembled microfluidic device into 3 lanes that become intravascular (I), vessel wall (V), and extravascular (E) compartments (FIG. 1D). Collagen mixed with lipidated tissue factor is loaded and polymerized in the middle lane of the device. (FIG. 1E). A 3D projection image shows the homogeneous distribution of tissue factor (stained with annexin V; magenta) in the collagen gel (FIG. 1F). Endothelial cells are seeded directly on top of the exposed collagen gel to form a confluent monolayer (FIG. 1G). Sequential steps to create an injury in the device. The micrographs show the top view of the device as an acupuncture needle is inserted through the engineered vessel wall (FIG. 1H). Representative images and quantification of puncture injuries that result from the insertion of 120 pm (small) and 200 pm (large) needles (FIG. 1I). FIG. 1J shows representative images of injuries with different penetration depths: 200 pm (puncture) and 100 pm (superficial).

The design of the system, characterized by three interconnected parallel microchambers, provided for emulating the physiological compartmentalization of vascular tissue. The middle chamber was configured to house a 3D collagen hydrogel containing homogeneously distributed tissue factor to model the deformable, procoagulant wall of a blood vessel (e.g., Vessel wall in FIG. 1C). The microchannel along the right side of this microengineered vessel wall was used to represent the intravascular compartment in which the human vascular endothelium was generated on the exposed hydrogel surface and perfused with whole blood at a venous shear rate to recapitulate the hemodynamic environment of the native vascular system (e.g., Intravascular channel in FIG. 1C). The left microchannel served as an extravascular compartment into which blood can escape when a hole is punched through the vessel wall (e.g., Extravascular channel in FIG. 1C).

To construct the disclosed device, a pair of identical PDMS microchannels were generated, each of which consisted of three parallel lanes separated by two thin microfabricated rails running along the length of the channel (FIG. 1D). Alignment and permanent bonding of the two-channel layers produced a sealed microdevice containing a microchamber with cross-sectional dimensions of 1 mm (width)×1 mm (height) flanked by two microchannels that measured 500 gm (width)×1 mm (height) (FIG. 1D). The middle chamber was open to the side channels through the gap between the upper and lower rails, but the three compartments were equipped with their dedicated access ports for independent fluidic control.

Model construction in this device started with the injection of a collagen hydrogel precursor solution mixed with lipidated tissue factor into the middle chamber. During this step, the injected solution was pinned at the microfabricated rails by surface tension, which effectively prevented the spillage of liquid into the side channels (Figure E). Because of the pinning effect, the solution remained confined stably between the rails while advancing along the entire length of the chamber (Figure E). After gelation, the middle chamber was entirely filled with a tissue factor-laden collagen hydrogel firmly anchored to the PDMS surfaces functionalized for strong interfacial adhesion with long-term stability (FIGS. 1E and 1F). The culture of primary human umbilical vein endothelial cells (HUVECs) on the exposed collagen surface led to the formation of an endothelial barrier with structural integrity (FIG. 1G).

Simulation of penetrating vascular injury in the blood vessel-on-a-chip: The disclosed compartmentalized microphysiological platform was then further engineered for in vitro modeling of bleeding due to penetrating vascular injury. A technique to interface the microengineered vascular tissue inside the disclosed device directly with a microneedle that can be controlled with high spatial precision from the macroscopic external environment was used (FIG. 1H). A cylindrical access port was created sideways between the two halves of the device to permit direct entry into our vascular model (FIG. 1H). To create the injury, a flexible needle was inserted through the rigid access port and guided into the intravascular channel (FIG. 1H), piercing the endothelial barrier and the collagen hydrogel construct and reaching the extravascular channel on the other side (FIG. 1H). Withdrawing the needle generated an open puncture wound across the vessel wall (Figure II).

The disclosed system demonstrated the feasibility of controlling the physical characteristics of vascular injury generated by this technique. For example, the size of the endothelial injury was readily changed by changing the diameter of the needle (FIG. 1I). However, the collagen matrix comprising the vessel wall relaxed when the needle was withdrawn, resulting in a 20-30% reduction in the size of the injury (FIG. 1I). Injury depth can also be controlled, which in turn can be conducive to modeling injuries of different severity. This capability was demonstrated by creating fully-penetrant injuries spanning the entire vascular construct and superficial injuries that penetrated only 25% of the full thickness of the vessel wall (FIG. 1J). In either case, needle penetration was accomplished with precision and reproducibility, as illustrated by less than 15% variation in the depth of injury (FIG. 1J).

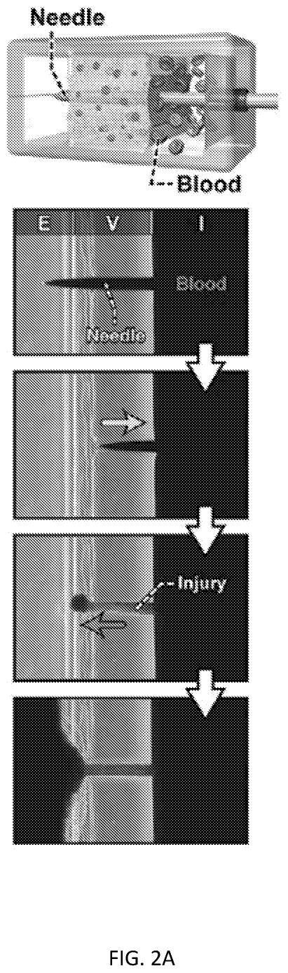

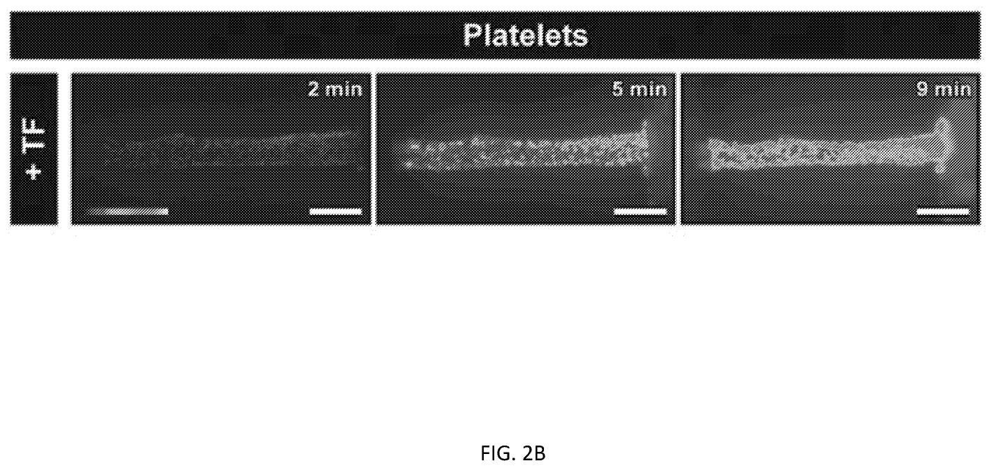

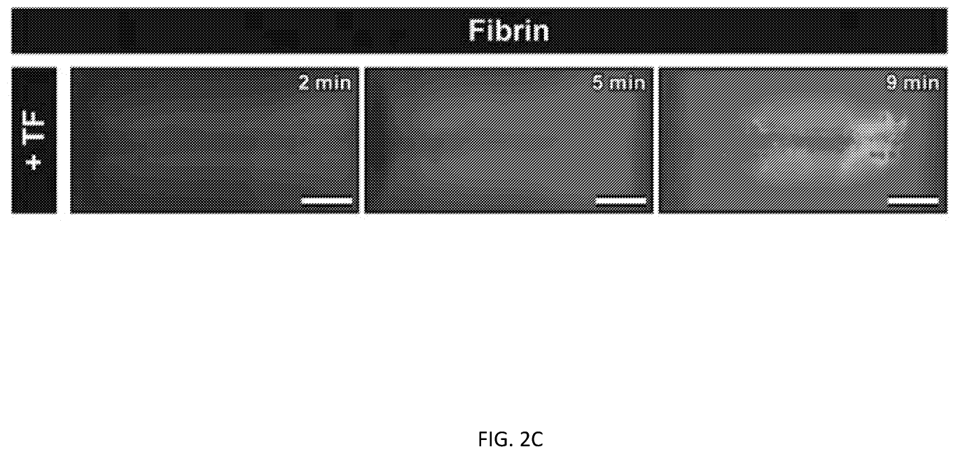

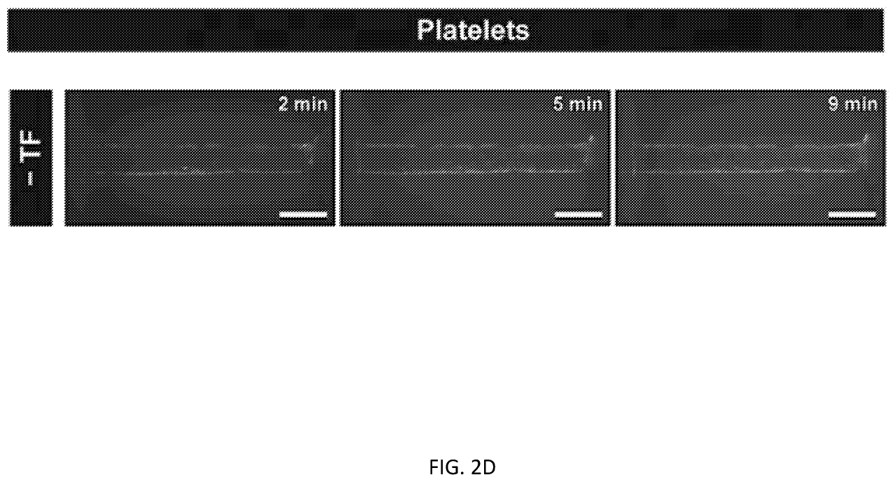

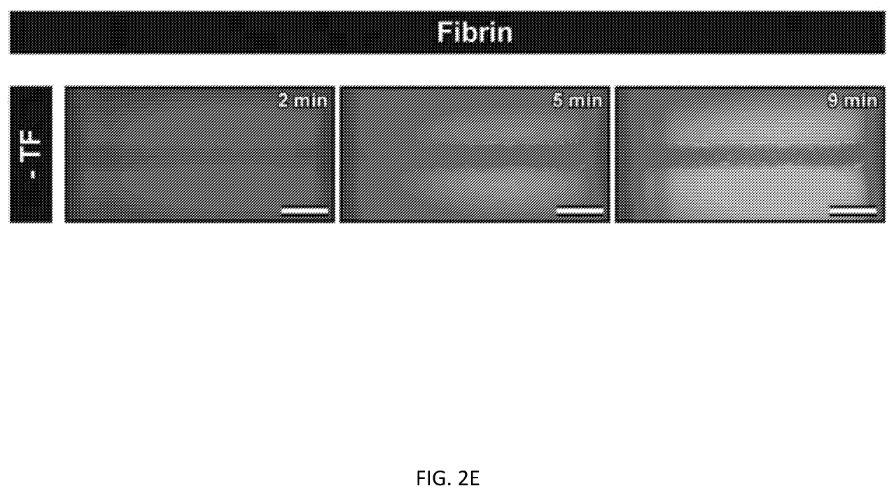

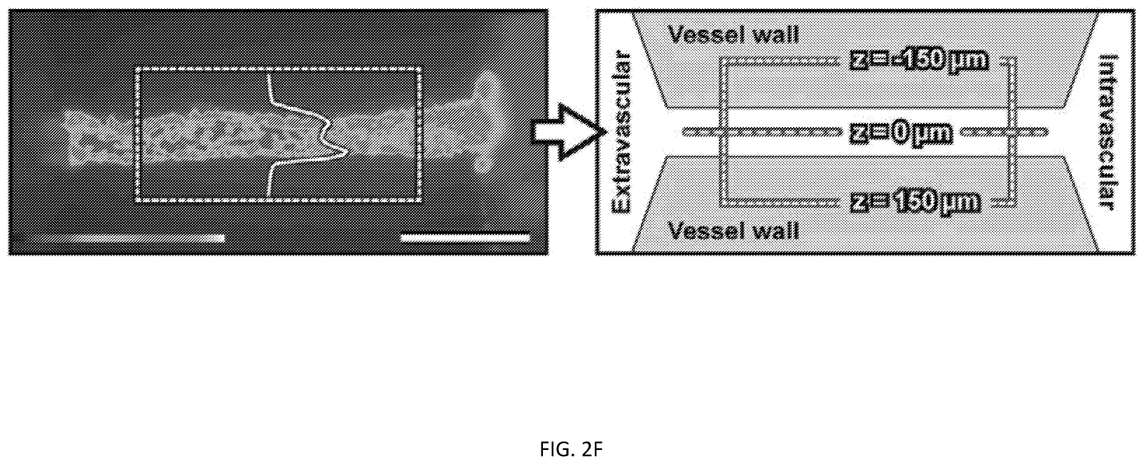

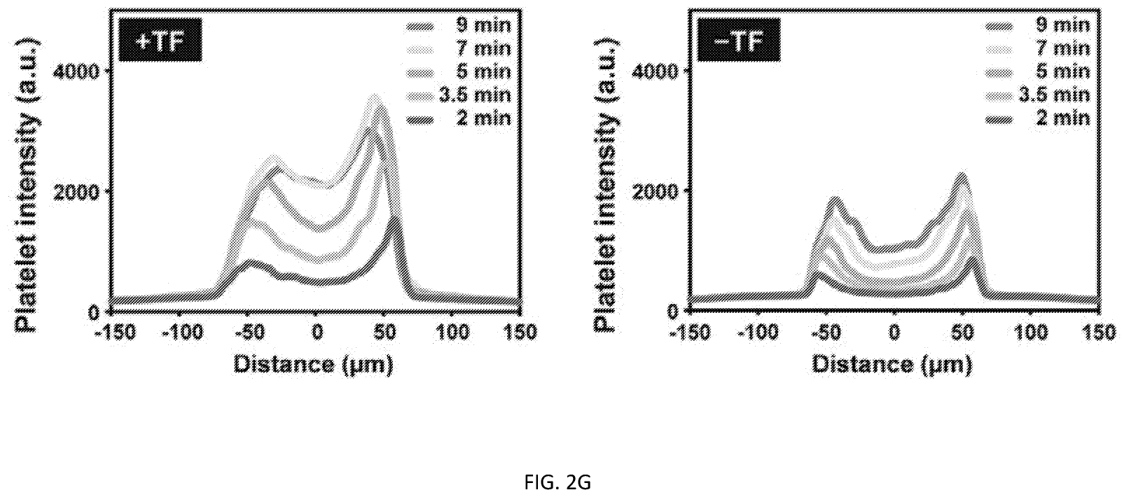

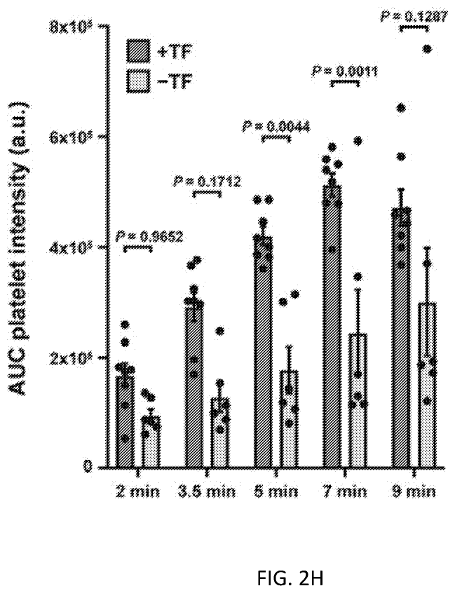

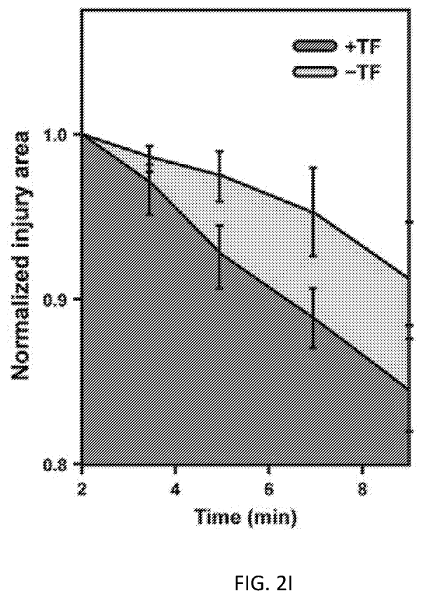

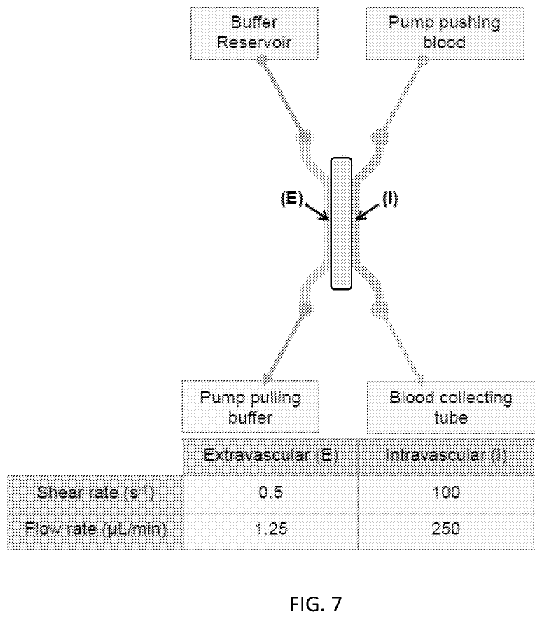

Formation of hemostatic plugs following injury: to assess hemostatic plug formation, the intravascular channel was perfused with human whole blood recalcified in the presence of corn trypsin inhibitor at a venous shear rate of 100 s⁻¹, while a buffer solution was pulled through the extravascular channel at 0.5 s⁻¹. When the vessel wall was punctured with the needle, the difference in the rates of flow in the intravascular and extravascular channels produced a pressure drop across the vascular construct, promoting the blood in the intravascular compartment to escape into the extravascular channel (FIGS. 2A and 7).

FIGS. 2A-2I shows the formation of platelet and fibrin-rich hemostatic plugs after a puncture injury. In the microdevice, bleeding is modeled as the leakage of blood through the injury due to the pressure difference between the intravascular (I) and extravascular (E) channels (FIG. 2A). Representative images of platelet deposition (Figures B and D) and fibrin accumulation (Figures C and E) in the presence (top row) and absence (bottom row) of tissue factor (TF) are shown. The scale in 2B and 2D indicates the number of platelets. The mark in 2C and 2E shows fluorescence emitted by the anti-fibrin antibody. In the presence of TF (FIG. 2C), fibrin deposits are detected as aggregates with intense green fluorescence in the injury channel at 9 min. The diffuse green haze seen in the matrix, particularly in the absence of TF, is due to the absorption of the fluorescently-tagged anti-fibrin antibody into the hydrogel vessel wall. FIG. 2F shows a region of interest in the vessel wall (shown with dotted lines) for microfluorimetric analysis of platelet deposition across the width of the injury. FIG. 2G shows a line scan of fluorescence intensity averaged over the length of the injury channel. FIG. 2G shows a plot of the area under the curves in g over time. FIG. 2I shows quantification of the change in the area of the 2D view of the injury shows greater matrix contraction over time in the presence of TF.

The optical transparency of the disclosed device enables direct observation of this dynamic process of bleeding and measure the kinetics of platelet accumulation. For real-time imaging, fluorescently-labeled antibodies to CD61, P-selectin, and fibrin were added to the blood perfused through the microfluidic device. Prior to the injury, no platelet adhesion was observed in the intravascular channel, illustrating the role of the intact endothelial barrier in preventing thrombotic events. Within a few minutes of injury, however, platelets adhered to the surface of the exposed collagen matrix and began to form small aggregates over time (FIG. 2B). Most of these deposits were initially observed at the ends of the puncture, but with time, platelet accumulation became pronounced and extended throughout the injury as the aggregates grew larger. Although the leakage of unbound fibrin antibody into the collagen matrix generated a background signal in the vessel wall, fibrin deposition was clearly visible as filamentous structures within the injury channel (FIG. 2C). This simultaneous accumulation of platelet and fibrin produced hemostatic plugs that halted blood loss within 10 minutes of the injury.

After confirming the ability of the disclosed model to generate hemostatic plugs, the device was used to confirm whether the lipidated tissue factor embedded in the collagen matrix influenced the hemostatic response. When the experiments were conducted in the absence of tissue factor, drastically reduced platelet and fibrin accumulation at the injury site were observed (FIG. 2D and 2E). To examine this difference quantitatively, the spatiotemporal distribution of platelet deposition was analyzed by selecting a region of interest within the injured vessel wall (FIG. 2F) and measuring the intensity profile of platelets over time. Regardless of whether tissue factor was present, the initial deposits occurred at the edges of the injury (FIG. 2G). However, when the collagen hydrogel contained tissue factor, this localized event propagated towards the center of the injury over a short period of time, leading to a rapid, substantial increase in fluorescence intensity across the entire width of the puncture (FIG. 2G; left). This spatiotemporal pattern of platelet deposition was significantly altered by leaving out tissue factor. In this case, the intensity increase was markedly reduced, and changes in fluorescence occurred in a more localized manner along the edges of the injury (FIG. 2G; right).

Quantification of the area under the curve of the intensity profiles also showed that platelet accumulation occurred at a faster rate in the presence of tissue factor, reaching a peak within about 7 minutes of bleeding (FIG. 2H). After that time, the fluorescence signal did not change much, indicating any further platelet deposition. Rapid contraction of the tissue factor-containing collagen matrix and a resultant decrease in the diameter of injury over the course of bleeding, which was evidenced by more than 15% reduction in the area of injury by the time of wound closure, was observed (FIG. 2I). In the absence of tissue factor, matrix deformation occurred to a much lesser extent (FIG. 2I).

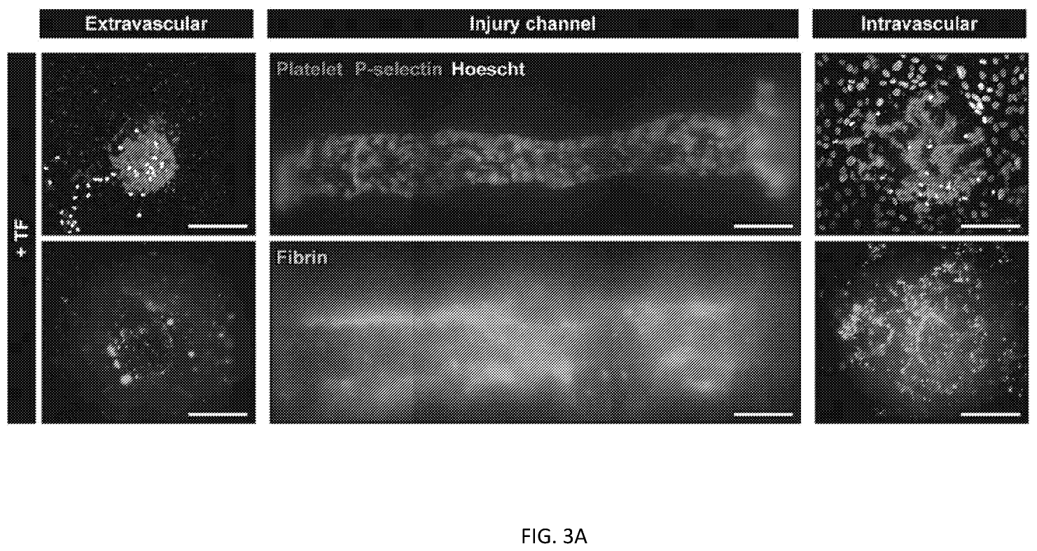

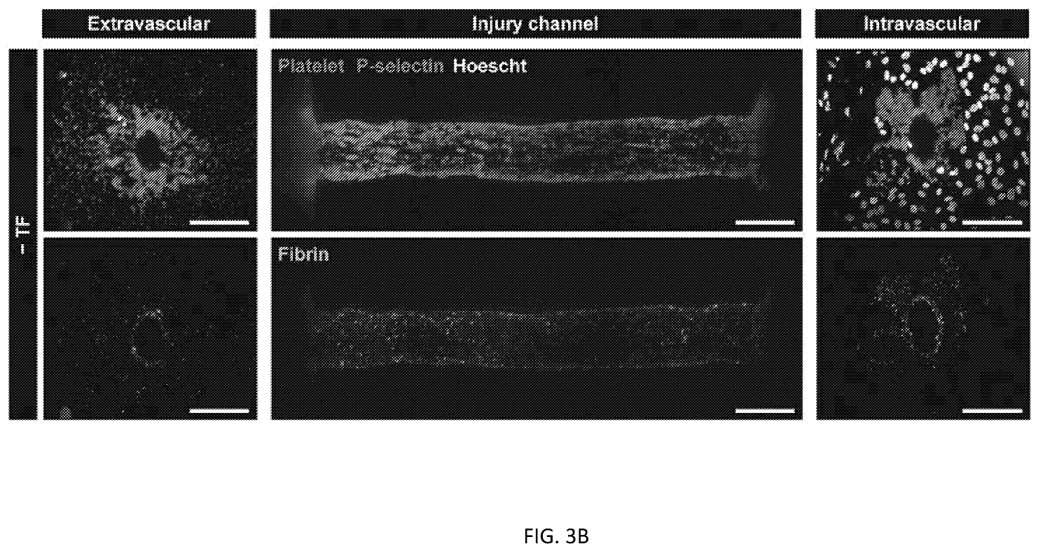

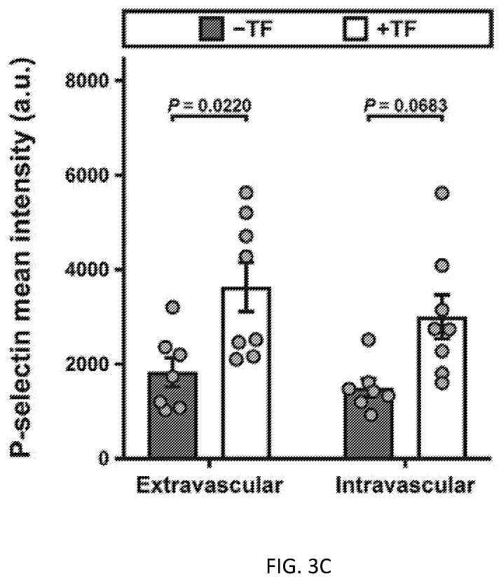

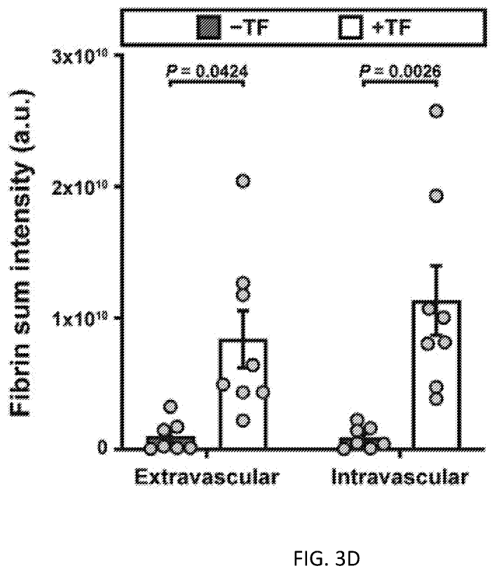

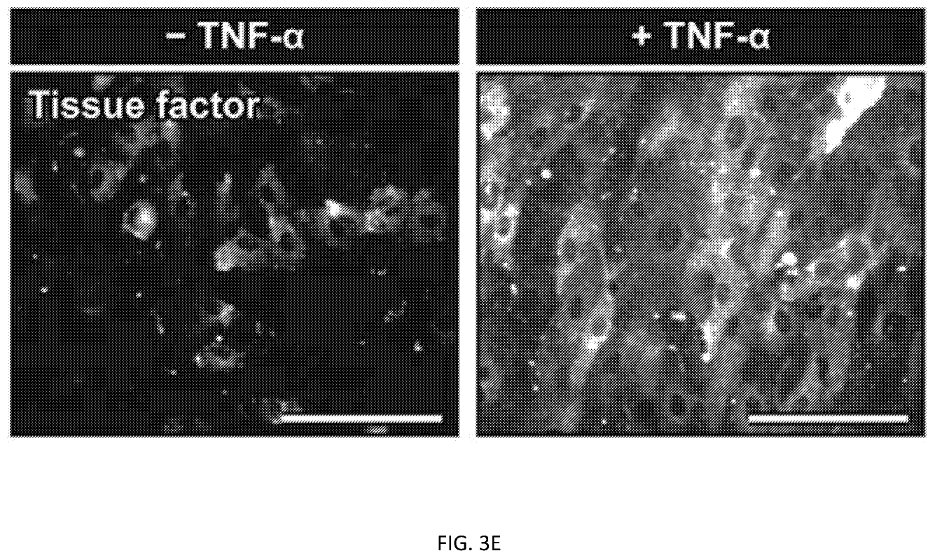

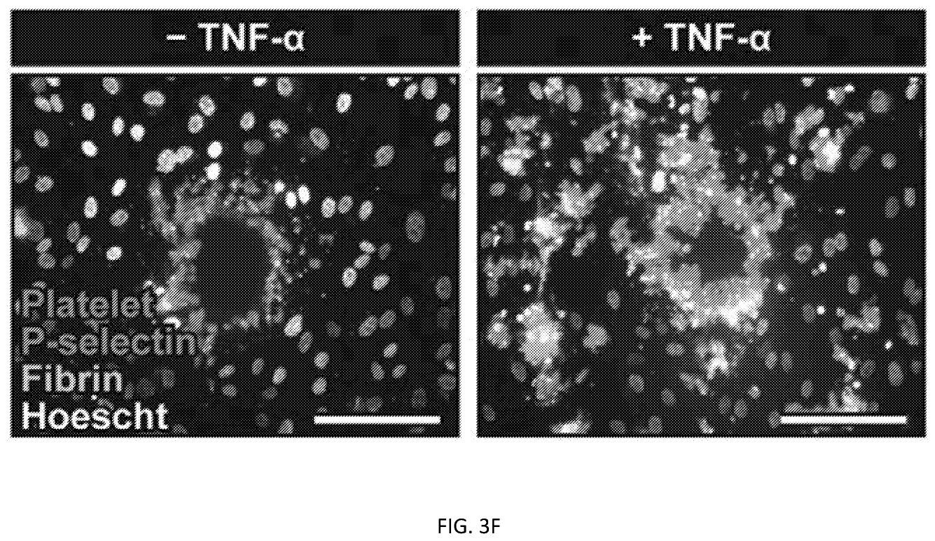

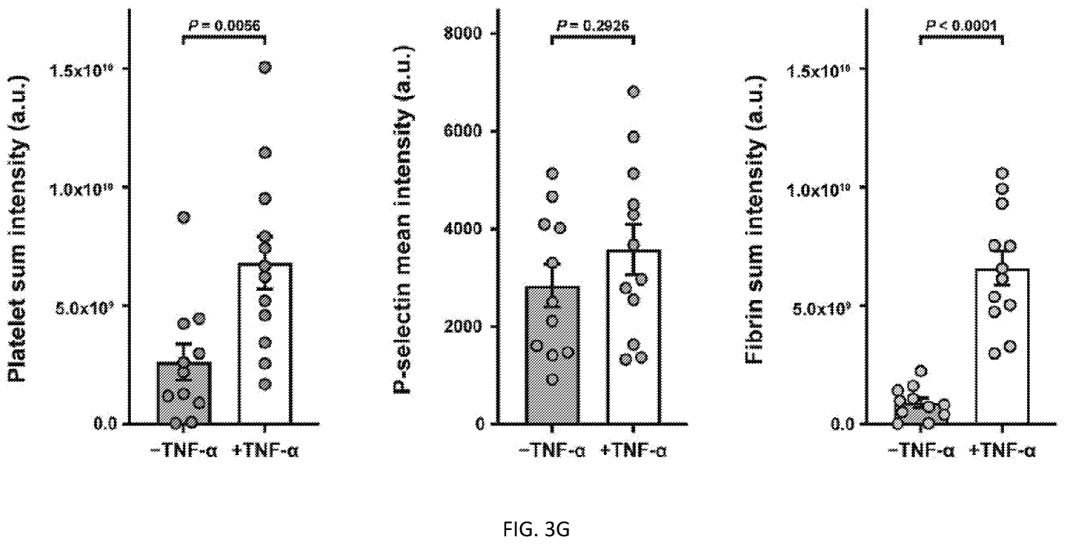

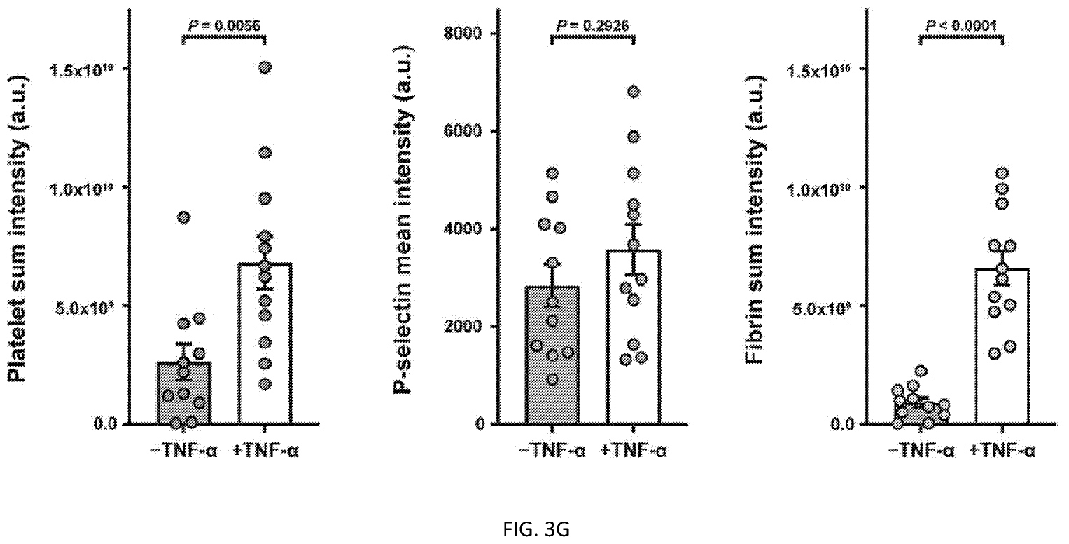

Platelet activation and fibrin formation: to further characterize the structure of hemostatic plugs in our microfluidic system, confocal microscopy was used to examine the spatial distribution of activated platelets and fibrin after bleeding through the injury channel stopped. Consistent with the findings of real-time imaging analysis described in FIGS. 2A-2I, maximum intensity projection images showed considerably increased platelet accumulation, P-selectin expression, and fibrin formation due to the presence of tissue factor in the microengineered vascular construct (FIGS. 3A-3G).

FIGS. 3A-3G show the characterization of platelet activation and fibrin formation. FIGS. 3A and 3B show maximum projection confocal images of the injury opening on the extravascular (left) and intravascular (right) sides. The middle column shows the top-down view of the injury in the vessel wall. Immunostaining of platelets, P-selectin+platelets, fibrin, and cell nuclei are shown. Quantification of the mean fluorescence intensity (MFI) of P-selectin (FIG. 3C) and the sum intensity of fibrin (FIG. 3D) from a 40 pm-thick z-stack. The results indicate an increase in intravascular platelet activation and fibrin accumulation in the presence of TF and an increase in fibrin at the extravascular end of the channel (3 donors; +TF: 8 devices −TF: 6 devices). Devices were perfused overnight with buffer ±TNF-α (1 ng/ml) prior to the start of the experiment (FIGS. 3E-3G). The tissue factor was not embedded in the collagen gel. Confocal images show increased TF expression in endothelial cells after TNF-α treatment (FIG. 3E). Confocal images obtained from the intravascular side of the injury channel after blood perfusion illustrate an increase in platelet and fibrin accumulation in TNF-α-treated devices (FIG. 3F). FIG. 3G shows quantification of platelet and fibrin sum intensity and P-selectin MFI (4 donors; +TNFα: 11 devices, −TNFα: 10 devices).

Analysis of hemostatic plugs in this procoagulant model revealed the following characteristics. First, the puncture injury was completely clogged with platelets, which was best visualized by the cross-sectional image of the injury opening on the extravascular side—the vast majority of platelets were found in the hole (FIG. 3A). Platelets were more widely distributed around the intravascular end of the injury to form a plug-like structure that sealed the breach (FIG. 3A). Second, most of the P-selectin-positive platelets were detected at the edges of the injury channel (FIG. 3A). The deposition of fibrin occurred in a similarly localized fashion and was observed mainly along the exposed surface of the injury within the vessel wall (FIG. 3A). The disclosed data also revealed different microscopic features of hemostatic responses in the absence of tissue factor. The difference was immediately apparent from the failure of wound closure. A number of adherent platelets were observed at both the intravascular and extravascular ends of the injury, but they accumulated only in the vicinity of the puncture without filling the openings (FIG. 3B). Similarly, platelet deposition within the vessel wall was confined to the surface of the injury channel (FIG. 3B). Another feature distinct from the tissue factor-containing model was the significantly decreased extent of platelet activation (FIG. 3C) and fibrin accumulation, which was almost negligible (FIG. 3D).

Taken together, these results showed the critical role of tissue factor in hemostasis of the disclosed vascular injury model. The disclosed technique was further used to test whether the location of tissue factor influences the formation of competent hemostatic plugs in our system. A condition in which tissue factor was localized on the endothelial lining of the intravascular channel was created, rather than being embedded in the collagen matrix by treating the endothelium with tumor necrosis factor (TNF)-α, which is a known inducer of endothelial tissue factor expression. Overnight treatment with 1 ng/ml of TNF-α resulted in robust and widespread expression of tissue factor in the endothelial cells (FIG. 3E). Bleeding caused by the puncture injury in this model triggered the hemostatic response and led to the aggregation of platelets at the injury site (FIG. 3F). In comparison to the control group that did not receive TNF-α, substantially increased deposition and activation of platelets over a larger surface area, which was accompanied by more pronounced fibrin production and accumulation, were observed (FIG. 3F). The increased thrombotic activities can be explained by the high levels of tissue factor present on the TNF-α-treated endothelial surface. The enhanced hemostatic response in this system, however, still failed to form plugs necessary to seal the opening of the injury (FIG. 3F), providing further evidence that tissue factor situated in the subendothelial compartment of the blood vessel wall is essential for proper hemostasis.

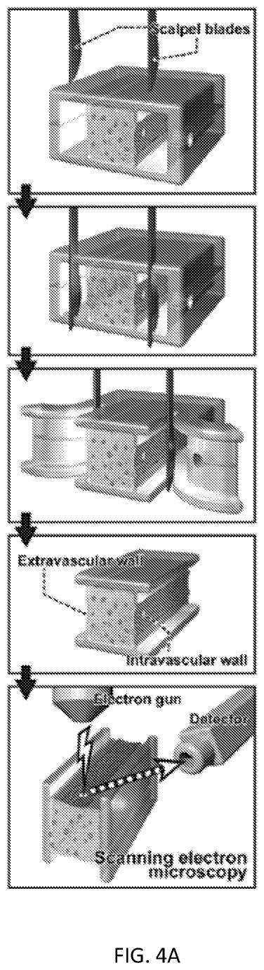

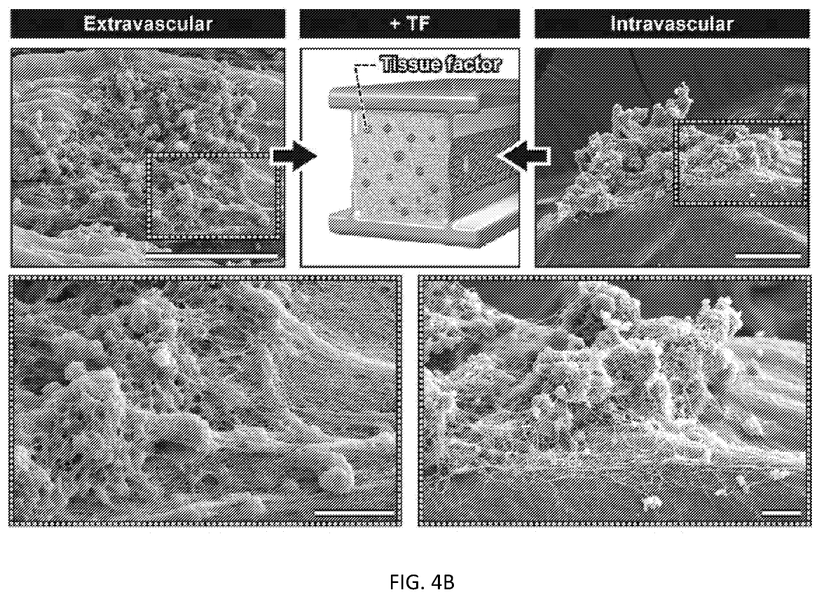

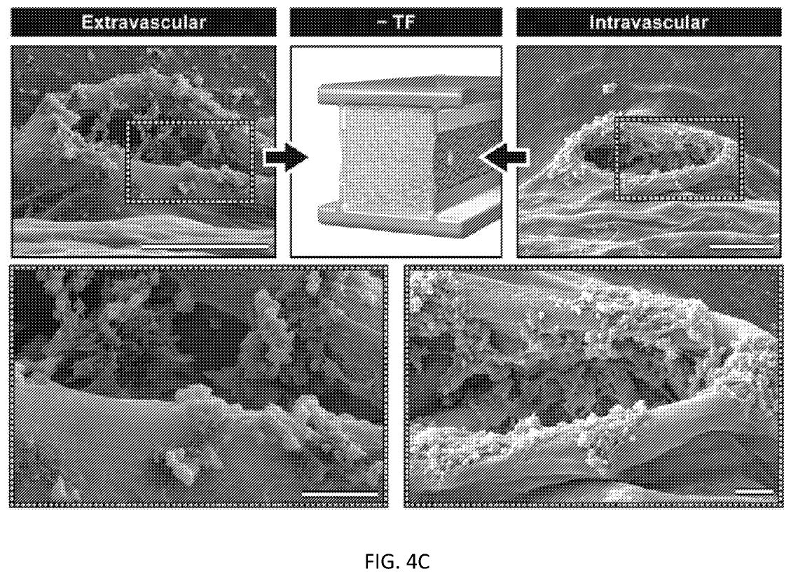

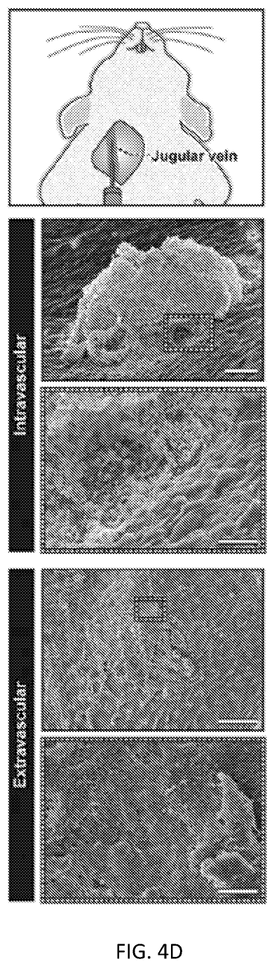

Direct visualization of hemostatic plugs for in vitro-in vivo comparison: the disclosed techniques were further used to test the feasibility of gaining access to the microengineered vascular construct after injury in order to directly observe and probe the microarchitecture of hemostatic plugs. The disclosed method for the testing analogous to microsurgical techniques that rely on a microscopically guided and manipulated scalpel blade to excise the intravascular and extravascular compartments and expose the injured vessel wall (FIG. 4A). By making the entire tissue construct readily accessible from the external environment, this technique permits direct visualization of the intravascular and extravascular surfaces around the injury site using scanning electron microscopy (SEM).

FIGS. 4A-4D show in vitro-in vivo comparison of hemostatic plugs using SEM. Microblades are used to precisely excise the device and expose the intravascular and extravascular sides of the hemostatic plugs formed in the vascular injury-on-a-chip for morphological examination using SEM (FIG. 4A). In the TF-containing device, hemostatic plugs composed of platelets (spherical aggregates) and fibrin (fibrous meshwork) fill the holes in the intravascular and extravascular sides of the injury (FIG. 4B). Scale bars are 60 pm (top; lower magnification) and I 0 pm (bottom: higher magnification). In the absence of TF, platelets adhere to the exposed collagen but fail to fill the hole created by the injury (FIG. 4C). Scale bars are 60 pm (top; lower magnification) and 10 pm (bottom: higher magnification). Scanning electron micrographs obtained from a murine model of jugular vein injury show a large hemostatic plug and a dense network of platelets and fibrin at the intravascular and extravascular openings of the injury, respectively (FIG. 4D). Scale bars are 100 pm (intravascular; lower magnification) and 50 pm (intravascular; higher magnification), 300 pm (extravascular; lower magnification) and 30 pm (extravascular; higher magnification).

The SEM micrographs clearly showed a large number of platelets localized to the injury site, many of which formed 3D aggregates (FIG. 4B). These platelet deposits were entangled in a highly dense fibrous network of fibrin formed on the surface of the endothelium (FIG. 4B). These composite structures filled and completely covered the opening of the puncture injury on both the extravascular and intravascular sides (FIG. 4B), corroborating the findings of confocal microscopy (FIGS. 3A-3G). When tissue factor was not present in the wall, however, any plugs of similar size and architecture were not detected (FIG. 4C). Although small aggregates were seen along the edges of the injury opening, the hole created by piercing injury was not occupied by platelets or other components of blood and remained unobstructed (FIG. 4C).

To compare the hemostatic plugs in the vascular injury-on-a-chip with those formed in vivo, a mouse jugular vein was punctured with a 30-gauge needle, fixed and excised 5 minutes postinjury, and prepared for SEM. Consistent with the hemostatic response in our device, the micrographs showed a 3D aggregate of platelets on the intravascular side that filled the opening of the injury (FIG. 4D). In comparison to the disclosed model, the hemostatic plug was bigger, and no fibrin was visible in and around the aggregate (FIG. 4D). Closure of the injury also occurred on the extravascular side, but the opening was covered with a dense carpet of platelets and fibrin (FIG. 4D) similar to what was observed in the vascular injury-on-a-chip.

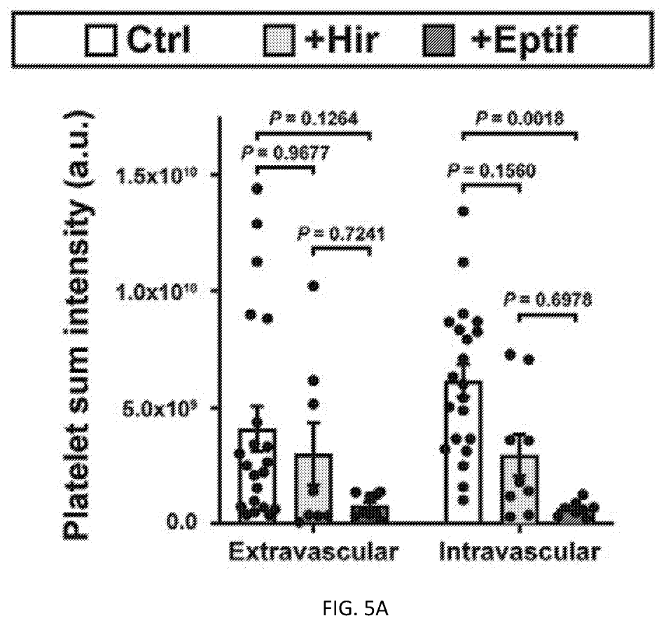

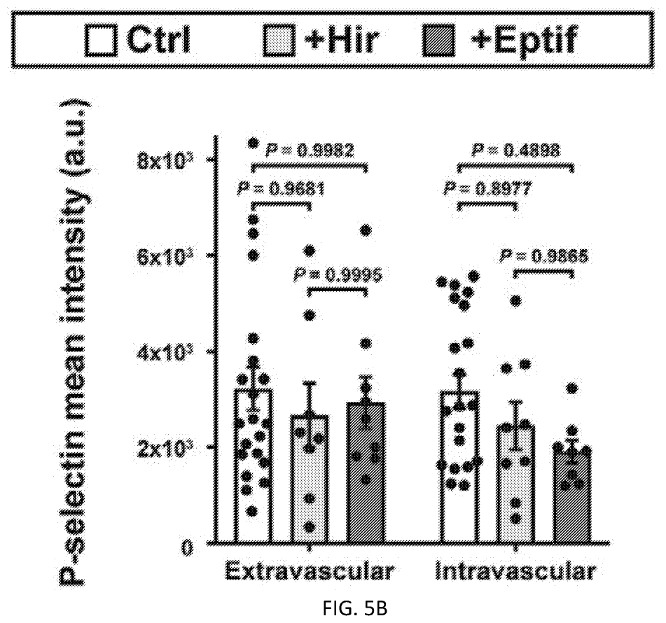

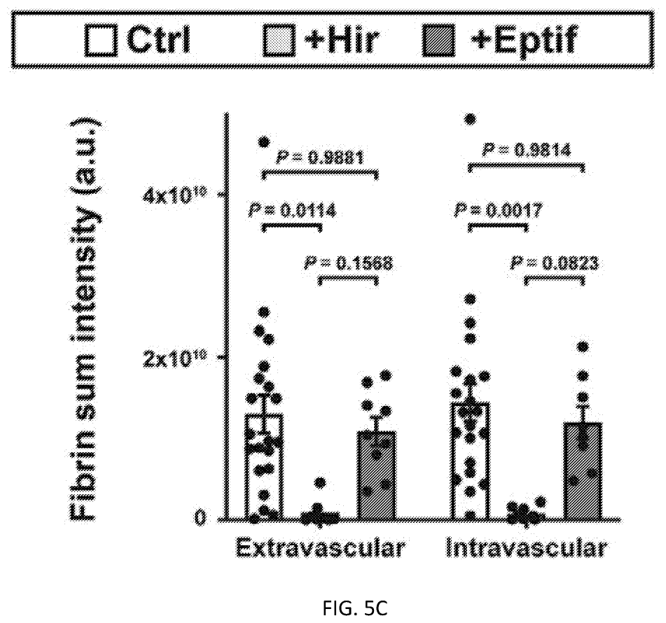

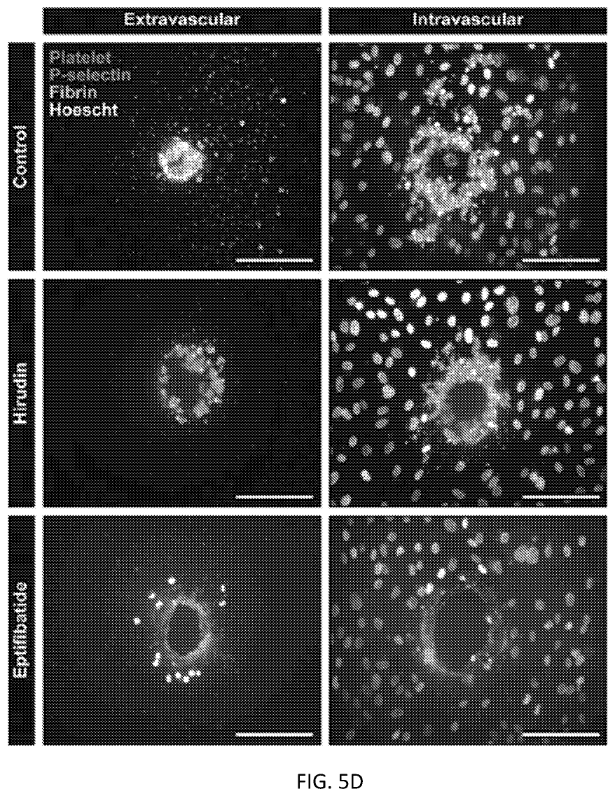

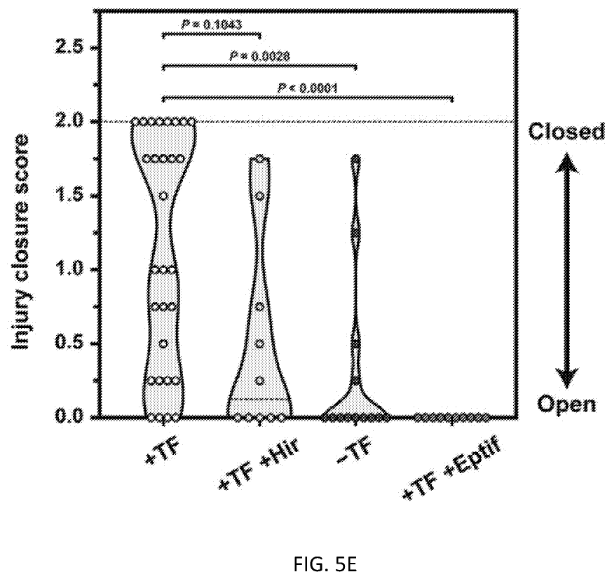

Human vascular injury-on-a-chip as a drug screening platform: anticoagulants and antiplatelet agents can be used for treating or preventing thrombotic events in the arterial and venous circulations. However, anticoagulants and antiplatelet agents can increase the risk of bleeding, especially when used in combination. The disclosed vascular injury-on-a-chip model provides a potential tool for evaluating these adverse effects, offering a complementary approach to animal models. Such features were tested using Hirudin and eptifibatide as model compounds representing anticoagulant and antiplatelet drugs, respectively. The disclosed subject matter was used to measure and compare the extent of injury-induced platelet and fibrin deposition when the disclosed devices were perfused with drug-containing whole blood. When Hirudin was administered at a clinically relevant concentration (e,g,m 4 μg/ml), the analysis showed no statistically significant changes in platelet accumulation and activation as compared to control without the drug (FIG. 5A and 5B). Hirudin did, however, drastically reduce fibrin accumulation, illustrating its efficacy as a thrombin inhibitor (FIG. 5C). Under this condition, the intravascular end of the injury channel remained open, but platelet aggregates blocked the extravascular opening (FIG. 5D). For quantitative analysis, we devised an injury closure score ranging between 0 and 2, which corresponds to fully open and fully closed, respectively. The hirudin-treated devices yielded an average score of while the untreated control group scored 1.138. (FIG. 5E), indicating the adverse effects of Hirudin on the formation of competent hemostatic plugs.

In contrast to Hirudin, the introduction of the antiplatelet agent, eptifibatide (e.g., 100 μm), did not change the extent of fibrin accumulation relative to control (FIG. 5C) but did markedly reduce platelet accumulation (FIGS. 5A and 5B). In this case, the injury channel remained open, as illustrated by its unobstructed ends (FIG. 5D) and an injury score of 0 (FIG. 5E). Failure of hemostasis in the eptifibatide-treated devices was further evidenced by negligible contraction of the vessel wall (FIG. 5F).

FIGS. 5A-5F shows drug testing in the vascular injury-on-a-chip. Quantification of platelet deposition (FIG. 5A), platelet activation (FIG. 5B), and fibrin accumulation (FIG. 5C) in the Hirudin- and Eptifibatide-treated devices (Control: 6 donors, 21 devices; Hirudin: 4 donors, 9 devices; Eptifibatide: 4 donors, 9 devices). FIG. 5D shows representative maximum projection confocal images of the extravascular (left) and intravascular (right) injury openings with no treatment (top), Hirudin (middle), and eptifibatide (bottom). FIG. 5E shows a violin plot of the injury channel closure scores obtained at the final time point (+TF: 8 donors, 29 devices; +Hir: 4 donors, 10 devices; −TF: 5 donors, 13 devices; +Eptif: 4 donors, 10 devices). FIG. 5F shows quantification of changes in normalized injury area over time as the assessment of platelet-driven matrix deformation (4 donors, +Eptif: 10 devices, −Eptif: 12 devices). The results show that in the disclosed model, eptifibatide causes a reduction in platelet accumulation, platelet activation, matrix deformation, and injury channel closure. Hirudin inhibits fibrin accumulation and injury closure.

The disclosed subject matter provides a novel bioengineering approach to emulating the human hemostatic response to injury without the use of animal models. By combining microengineering design principles with primary human cell culture, the disclosed subject matter demonstrated the feasibility of reverse-engineering the complex, dynamic process of hemostasis in human blood vessels. The disclosed microphysiological system was capable of mimicking the entire process of the hemostatic response from injury to bleeding to wound closure, all of which can be visualized in real-time. The disclosed system was also used as a vascular injury-on-a-chip for applications in drug testing.

The disclosed vascular injury-on-a-chip provides various advantages for modeling hemostasis in vitro. The disclosed system enables the integration of the key elements of the vascular system that can be important for hemostasis in a more seamless and comprehensive manner. The disclosed device permits the flow of human whole blood at physiological rates in a microchannel lined with primary human vascular endothelial cells that can be supported by a deformable hydrogel containing collagen and tissue factor to mimic two key components of the vessel wall. In this configuration, the living endothelium serves as a barrier between the perfused blood and the procoagulant subendothelial compartment, recreating elements of the structural organization and hemodynamic environment of native vessels. The disclosed model can be equipped with a controllable microneedle that can simulate an acute vascular injury, enabling replication and can replace the kinds of puncture injuries used in animal models of hemostasis. The needle injury can create a controlled breach in the vessel wall of physiologically-relevant size, across which a pressure drop helps to drive blood from the intravascular channel to the extravascular channel. Along the way, the escaping blood comes into contact with collagen and tissue factor, triggering platelet adhesion, platelet activation, the localized generation of thrombin, and the formation of a hemostatic plug. At the same time, the deformable vessel wall can retract a process that is driven by activated platelets and contributes to the closure of injury. The disclosed subject matter also provides a vascular injury-on-a-chip as a preclinical model to assess the impact of drugs on platelet accumulation and fibrin deposition. Hirudin inhibited fibrin formation was observed while exerting negligible effects on platelet accumulation. Eptifibatide, in contrast, reduced platelet accumulation with minimal changes in fibrin deposition (FIGS. 5A-5F). It also inhibited injury channel closure to a greater extent than Hirudin. Taken together, these findings show that the hemostatic response in the disclosed system depends on both platelet accumulation and fibrin deposition.

The disclosed device can be modified to increase the number of individually accessible parallel chambers/lanes, each of which can contain different materials to more faithfully mimic the multilayered microarchitecture of the blood vessel wall. The same approach can also achieve an in vivo-like spatial distribution of perivascular cells. The disclosed subject matter can recreate arterial conditions by changing hemodynamic parameters such as the flow rate. The disclosed system can be an automated model that can increase precision and reproducibility to simulate different injury dynamics.

One of the features that the disclosed subject matter can provide is the deformation of the vessel wall due to contractile forces generated by activated platelets. The ability to capture this aspect of hemostasis can lead to probing biophysical characteristics of the hemostatic response that cannot be tested in vivo, including clot consolidation, reinforcement, and stability. The disclosed system can also provide a useful platform to investigate whether and how the severity of injury influences the activity of different molecular pathways involved in hemostasis.

The design of the disclosed system (e.g., the 3D collagen hydrogel compartment) can provide the flexibility to incorporate additional cellular and acellular components to mimic the vessel wall more precisely and extend the disclosed system for in vitro modeling of pathophysiological situations. For example, pericytes and other cellular components (e.g., fibroblasts) of the subendothelial tissue can be incorporated to account for their contributions to hemostatic and thrombotic events. Modifying the amount of TF or incorporating other prothrombotic molecules (e.g., oxidated LDL) into the matrix in conjunction with cellular components known to play a role in plaque formation can serve as a model to test thrombus formation after plaque rupture. The mechanical properties of the scaffold can be modified in order to model hemostasis and thrombosis in blood vessels with altered tissue mechanics due to vascular diseases (e.g., atherosclerosis). These adaptations and improvements can extend the application of our microphysiological system and potentially make important contributions to developing novel platforms for more reliable preclinical drug screening.

The disclosed subject matter provides an advanced in vitro technology realized by a biomimetic microengineering approach. The disclosed system can be a physiologically relevant and predictive model of hemostasis in the human vascular system. The disclosed vascular injury-on-a-chip model can provide a tool for emulating and probing the inner workings of the hemostatic response.

Materials: The Sylgard® 184 silicone elastomer kit was purchased from Dow Corning. Collagen I, high concentration, rat tail was purchased from Corning; Dade® Innovin® reagent, from Siemens; and corn trypsin inhibitor (CTI), from Haematologic Technologies. Anti-CD61 (VI-PL2) antibody was purchased from BD Pharmingen, anti-P-selectin (AK4) antibody was purchased from Biolegend, and an anti-fibrin antibody that does not bind to fibrinogen was a gift. The antibodies were fluorescently labeled using the Alexa Fluor™ antibody labeling kits (488, 568, and 647) from Life Technologies, according to the manufacturer's instructions. Eptifibatide acetate and dopamine hydrochloride were purchased from Sigma-Aldrich. Hirudin was obtained from Profacgen (HY0073HL). Annexin V was a gift (Children's Hospital of Philadelphia) and was labeled. TNFa was purchased from Peprotech (300-01A). Human coagulation factor III/Utissue factor antibody was purchased from R&D Systems (AF2339), Alexa Fluor 647 mouse anti-human CD31 (clone WM59) was purchased from BD Pharmingen (561654), and 1CAM-1 was purchased from Biolegend (353102).

Microfluidic device fabrication: SU-8 masters containing the microfluidic channel features were prepared by conventional photolithographic techniques and used to fabricate our blood vessel-on-a-chip devices. Poly(dimethylsiloxane) (PDMS) base (Sylgard 184, Dow Corning) was mixed with a curing agent at a weight ratio of 10:1, degassed to remove air bubbles, and poured onto the masters. After baking at 65° C. for 3 hours, the cured polymer was peeled from the masters to generate two PDMS layers embossed with microchannel features. In one of the layers, inlet and outlet ports were made using a biopsy punch to gain fluidic access to the microchannels. For device assembly, the micropatterned PDMS slabs were treated with air plasma generated by a corona treater (ELECTRO-TECHNIC PRODUCTS, BD-20A), bonded after alignment of the channel features using light microscopy, and incubated at 65° C. overnight for complete bonding. Subsequently, medium reservoirs were attached to the upper microchannel slab using the same bonding technique described above. In the assembled device, the intravascular and extravascular channels had cross-sectional dimensions of 0.5 mm (width)×1 mm (height), whereas the middle compartment representing the vessel wall was 1 mm (width)×1 mm (height) (FIG. 6C).

Cell culture: Human Umbilical Vein Endothelial Cells (HUVECs), purchased from Lonza, were cultured in EGM-2 (CC-3162, Lonza) in 37° C. incubator with 5% CO2. HUVECs were used between passages 3 and 5 for device culture.

Preparation of blood vessel-on-a-chip: Assembled devices were autoclaved for sterilization prior to cell culture. To increase the strength of interfacial adhesion between collagen gel and PDMS, dopamine hydrochloride (Sigma-Aldrich) solution (2.0 mg/mL w/v in 10 mM Tris-HCl buffer, pH 8.5) was injected into the middle compartment of the device and incubated for 2 hours at room temperature. Next, 6 mg/ml of rat-tail collagen type I containing 2.5% (v/v) lipidated tissue factor (Dade Innovin Reagent, Siemens) was introduced into the polydopamine-treated chamber and polymerized at 37° C. for 30 minutes. After gelation, HUVECs suspended in EGM-2 medium were seeded into the intravascular channel at a density of 1.6-1.8×106 cells/ml and allowed to settle and adhere to the surface of the collagen hydrogel by rotating the device by 90°. Following cell attachment, the device was perfused with medium to form a confluent endothelial monolayer. For TNF-a experiments, HUVECs were seeded on a tissue factor-free collagen gel and treated overnight with 1 ng/ml of TNF-a (Peprotech).

Healthy donor blood preparation: The blood was obtained via venipuncture into a syringe containing 3.8% sodium citrate (9:1) and 40 μg/ml of corn trypsin inhibitor (CTI). Right before perfusion through the device, the blood was recalcified (15 mM CaCl2) for 3 minutes. Fluorescently-labeled antibodies to detect platelets (a-CD61-568), P-selectin (a-CD62P-647), and fibrin (a-Fibrin-488) were added at a final concentration of 1 μg/ml. If inhibitors were used in the experiment, they were added in the recalcification step as well. The final concentration for eptifibatide acetate was 100 μM, and for Hirudin was 4 μg/ml.

Puncture injury and global hemostasis assessment: For precise spatial control of injury, a 30 G blunt needle was inserted between the two PM DS channel layers during device assembly and used as a sheath that guides the insertion of a microneedle. To induce puncture injury, either a 0.12 or 0.20 mm acupuncture needle was inserted into the guide needle and gently pushed towards the extravascular compartments in a controlled manner either to pierce through the endothelium and the entire thickness of the collagen hydrogel or to superficially injure the endothelial layer. Following injury, the intravascular and extravascular channels were perfused with recalcified blood and HBSS buffer (20 mM HEPES, 2 mM Ca2+), respectively using two independently controlled syringe pumps (Fusion 400, Chemyx & BS-8000 120 V, Braintree Scientific).

Fluorescently-labeled antibodies to CD61, P-selectin, and fibrin were added to the blood prior to perfusion through the microfluidic device to visualize platelet deposition, platelet activation, and fibrin accumulation. Different biomarkers of the hemostatic response, such as platelet accumulation and fibrin formation at the injury site, were monitored using real-time fluorescence imaging using the Zeiss Axio Zoom.V16, objective PlanNeoFluor Z 1.0×, with a total 75×, using the Axiocam 506 camera. For real-time analysis, images were captured every 10 seconds for 10 minutes (FIG. 1C). After perfusion, the device was fixed and imaged with a 20×water-immersion objective and a CSU-X1 spinning disk confocal scanner (Yokogawa). Z-stacks with 2 μm steps were captured with an Evolve digital camera (Photometrics).

Immunofluorescence staining: For immunostaining, devices were fixed at room temperature for at least 10 minutes with 4% paraformaldehyde and washed three times with PBS. For tissue factor staining, devices were blocked with 3% bovine serum albumin for 1 hour, followed by overnight incubation with primary antibody (AF2339, R&D Systems, 1:20 dilution) at 4° C. Subsequently, devices were washed three times, 10 minutes per wash. Secondary antibody incubation was conducted for 1 hour at room temperature (Alexa Fluor 488 anti-goat, A11078, Life Technologies). As specified by the manufacturer, the cells were incubated at 37° C. for 30 minutes at a 1:50 dilution, followed by three buffer washes. After immunostaining, cells were incubated with 1 μg/ml of Hoescht solution (33342, ThermoFisher) in HBS for 10 minutes at room temperature to visualize the nuclei of HUVECs, which was followed by buffer washes.

Analysis of injury diameter and depth: Phase contrast images of the intravascular opening of the injury were used to measure the injury diameter. In a given image, two perpendicular lines were drawn across the opening that intersected at the center of the hole. The diameter of the injury was calculated as the average length of these lines. To quantify the depth, we used the longitudinal sections of the injury (FIG. 1G) to measure the length of the centerline of the injury channel from the intravascular opening to the other end.

Quantification of rectangular intensity profile: Lines that defined the boundaries of the collagen gel were drawn to determine the center of the region of interest for analysis. To account for deformation of the collagen gel, the boundaries were redefined for each time point. Information was extracted for 5-time points: 2, 3.5, 5, 7, and 9 minutes. A rectangle intensity profile (600 pm wide by 360 pm high) was created for the platelet channel for each time point. The curves of the intensity profile went from top to bottom across the length of the 360 pm height of the rectangle (FIG. 2F). For background correction, the maximum intensity of the first 50 pm was subtracted from all the values, and then 100 arbitrary units were added to shift the curve above zero. The curves were aligned to make zero the center of the hole/injury and cropped so the curves can span from −150 to +150 pm. From that range, the area under the curve (sum of MFI's for each time point) was calculated from −75 to +75 pm and plotted on a bar graph for each time point (FIG. 2H).

Measurement of sum and mean fluorescence intensity: For each image capture, 80 pm of the intravascular and extravascular side of the injury was analyzed—40 pm below the gel boundary and 40 pm above. For each channel, a mask was created from a threshold value that was defined from the average background intensity of the images. The total intensity was quantified, and for P-selectin, the mean fluorescence intensity (MFI) within the platelet mask was calculated for the total volume that was analyzed. Results for platelets and fibrin are shown as sum intensity and for P-selectin as MFIs (FIGS. 3A-3G and 5A-5F).

Quantification of matrix deformation: The deformation of the collagen matrix was evaluated by measuring changes in the injury area over time. This analysis was conducted at 2, 3.5, 5, 7, and 9 minutes after injury. For each time point, an injury area was defined by outlining the boundaries of the platelet signal within the channel, after which a mask was created for quantification of the area (FIGS. 2I and 5H).

Assessment of injury score: Injuries were scored from 0 to 2 with 0, 1, and 2 being fully open, partially closed, and fully closed, respectively. Open injuries were scored as those in which the platelet movement was continuous within the injury channel over the 10-minute period of observation. Partially closed injuries were defined as the injuries in which platelets were still moving but at slower rates than when the injury was fully open. For closed injuries, no platelet movement can be tracked in the injury channel. Following these general criteria, four different subjects blindly scored the injuries, and the average score was plotted for all of the injuries captured (FIG. 5E).

Murine model of vascular injury: A puncture injury to the mouse jugular vein was performed. Briefly, the right jugular vein was exposed and punctured using a 30-gauge needle (300 μm diameter). Extravasated blood was rinsed by slow perfusion of normal saline. The hemostatic response was stopped at 5 minutes via consecutive transcardiac perfusion of sodium cacodylate buffer (0.2 M sodium cacodylate, 0.15 M sodium chloride, pH 7.4) and 4% paraformaldehyde. The vein was excised, cut along its length, and pinned to a silicone pad, followed by preparation for SEM.

Scanning electron microscopy: Devices and mouse jugular veins were prepared for imaging by scanning electron microscopy with a sole difference being the use of sodium cacodylate buffered solution without NaCl for the devices to preserve the collagen structure better. Briefly, the samples were rinsed three times with the sodium cacodylate buffer, followed by serial dehydration with ethanol solutions going from 30% to 100%, double rinsed with hexamethyldisilazane, and left to dry overnight. A thin film of gold-palladium was deposited on the samples (Quorum Q 150T ES; Quorum Technologies), and micrographs were taken using a Quanta FEG250 scanning electron microscope (FIGS. 4A-4D).

Statistics: For each condition, we generated 6-8 devices and perfused them with whole blood collected from at least three donors. Data were presented as mean±SEM. Statistical tests were performed using Graphpad Prism 8. Two-way ANOVA and Tukey's multiple comparisons were conducted to determine statistical significance among multiple groups. Whenever the variance was significantly different, the Kruskal-Wallis test and the Dunn's multiple comparison test were used instead. Unpaired t-tests were carried out for comparison of two conditions in the quantification of fluorescence for different markers used. Welch's correction was applied to the unpaired t-test when needed.

All patents, patent applications, publications, product descriptions, and protocols, cited in this specification are hereby incorporated by reference in their entireties. In case of a conflict in terminology, the present disclosure controls.

While it will become apparent that the subject matter herein described is well calculated to achieve the benefits and advantages set forth above, the presently disclosed subject matter is not to be limited in scope by the specific embodiments described herein. It will be appreciated that the disclosed subject matter is susceptible to modification, variation, and change without departing from the spirit thereof. Those skilled in the art will recognize or be able to ascertain using no more than routine experimentation, many equivalents to the specific embodiments described herein. Such equivalents are intended to be encompassed by the following claims.

What is claimed:

1. A system, comprising:
a top layer including a plurality of top rails and a bottom layer including a plurality of bottom rails,
wherein the top layer and the bottom layer arc configured to be coupled,
wherein the plurality of top rails and bottom rails are configured to form a plurality of channels comprising:
an intravascular channel configured to circulate a first solution,
an extravascular channel configured to circulate a second solution, and
a vessel wall channel including a tissue factor in a hydrogel.

2. The system of claim 1, wherein the hydrogel comprises an endothelial cell for generating an endothelial monolayer.

3. The system of claim 2, wherein the endothelial cell is a human umbilical vein endothelial cell (HUVEC).

4. The system of claim 1, further comprising an inlet port and an outlet port for accessing the plurality of channels.

5. The system of claim 1, wherein the plurality of channels comprises a microchannel.

6. The system of claim 1, wherein the plurality of top rails and bottom rails arc microfabricated rails.

7. The system of claim 1, wherein the first solution comprises recalcified blood.

8. The system of claim 1, wherein the second solution comprises an HBSS buffer.

9. The system of claim 1, wherein the hydrogel is a collagen hydrogel.

10. The system of claim 2, wherein the system is configured to receive a needle that is inserted between the intravascular channel and the extravascular channel for generating puncture injury on the endothelial monolayer.

11. A method for drug screening, comprising:
seeding an endothelial cell in a hydrogel using a device comprising a plurality of channels comprising an intravascular channel, an extravascular channel, and a vessel wall channel, wherein the hydrogel is located in the vessel wall channel and comprises tissue factor;
culturing the endothelial cell by adding culture medium to the intravascular channel and the extravascular channel;
forming an endothelial monolayer in the vessel wall channel;
adding a target drug into the culture medium; and
measuring platelet deposition.

12. The method of claim 11, further comprising measuring fibrin deposition.

13. The method of claim 11, further comprising measuring platelet and fibrin deposition before adding the target drug.

14. The method of claim 13, further comprising comparing the platelet and fibrin deposition before adding the target drug with the measured platelet and fibrin deposition after adding the target drug.

15. The method of claim 11, wherein the target drug is selected from the group consisting of an anticoagulant drug, an antiplatelet drug, and a combination thereof.

16. The method of claim 11, wherein the endothelial cell is HUVEC.

17. The method of claim 11, wherein the target drug is added through an inlet port of the device.

18. The method of claim 11, wherein the hydrogel is a collagen hydrogel.

19. The method of claim 11, further comprising generating puncture injury on the endothelial monolayer.
